(19) United States
(12) Patent Application Publication
(10) Pub. No.: US 2011/0082119 A1
(43) Pub. Date: Apr. 7, 2011

(54) PROPHYLACTIC/AMELIORATING OR THERAPEUTIC AGENT FOR NON-ALCOHOLIC STEATOHEPATITIS

(76) Inventor: Takashi YANO, Shizuoka (JP)

(21) Appl. No.: 12/997,437

(22) PCT Filed: Jun. 12, 2009
(86) PCT No.: PCT/JP2009/060749

§ 371 Date: Dec. 10, 2010

(30) Foreign Application Priority Data
Jun. 13, 2008 (JP) 2008-156034
Jun. 13, 2008 (JP) 2008-156035
Jun. 13, 2008 (JP) 2008-156037
Jun. 13, 2008 (JP) 2008-156040

(51) Int. Cl. A61K 31/202; A61K 31/201; A61K 31/616; A61K 31/366; A61K 31/235; A61K 31/404; A61K 31/40; A61K 31/505; A61K 31/47; A61K 31/4418; A61K 31/20; A61K 31/4178; A61K 31/41; A61K 31/4184; A61P 1/16; A61P 29/00; A61P 3/10

(52) U.S. Cl. 514/163; 514/560; 514/460; 514/510; 514/419; 514/423; 514/275; 514/311; 514/277; 514/558; 514/381; 514/397; 514/394; 514/382

(57) ABSTRACT

A highly safe and effective prophylactic/ameliorating or therapeutic agent for NACH and the method for using the same are provided.

A prophylactic/ameliorating or therapeutic agent for NASH containing a combination of at least one first ingredient selected from the group consisting of an ω3PUFA and pharmaceutically acceptable salts and esters thereof and at least one second ingredient selected from the group consisting of (a) a biguanide hypoglycemic agent, (b) a nonsteroidal anti-inflammatory drug, (c) a 3-hydroxy-3-methyl glutaryl coenzyme A reductase inhibitor, and (d) an angiotensin II receptor blocker as the active ingredients; and its method of use.

### TECHNICAL FIELD

This invention relates to a prophylactic/ameliorating or therapeutic agent for non-alcoholic fatty liver disease, and in particular, non-alcoholic steatohepatitis. This invention also provides a method for using such agent.

### BACKGROUND ART

The group of diseases including the liver disorders from simple fatty acid to steatohepatitis, fibrosis, liver cirrhosis that occur in patients with no alcohol drinking history excluding viral liver disease, autoimmune liver disease, hemochromatosis, and metabolic liver diseases such as Wilson's disease are generically defined as non-alcoholic fatty liver diseases (hereinafter referred to as “NAFLDs”). The NAFLDs are further classified into simple fatty liver which is generally considered to have a favorable prognosis and non-alcoholic steatohepatitis (hereinafter referred to as “NASH”) with poor prognosis based on liver biopsy (pathological findings), and NASH is considered to be the serious version of the NAFLD. Pathological conditions from inflammation, fatty change, and fibrosis to liver cirrhosis and liver cancer which are diagnosed as NASH by the liver biopsy are the same as the pathological conditions induced by other causes, and many hepatitis patients who have been denied to be patients of alcoholic liver damage, viral hepatitis or drug-induced liver injury are estimated to have the pathological conditions of NASH (see Non-Patent Literature 1).

In the U.S., 20% of the population is estimated to suffer from the NAFLD, and 3% is estimated to suffer from the NASH. In Japan, NAFLD and NASH are also diseases that are frequently found in general practice, and frequency of the NAFLD in screening is 8%, and frequency of the NASH is estimated to be 0.5 to 1% of the adult population. For Japan, based on the number of obese adults with a BMI of not less than 25 of 13 million males and 10 million females, estimated number of the NAFLD patients is 5 to 6 million, and estimated number of the NASH is about 300 to 500 thousands. In the meanwhile, complication frequency of dyslipidemia in NAFLD patients based on diagnostic criterion of metabolic syndrome (hereinafter referred to as “MetS”) is about 50%, complication frequency of hypertension is about 30%, complication frequency of hyperglycemia is about 30%, and complication frequency of MetS is about 40% (see Non-Patent Literature 1), and increase of the NASH case and spread in the youth are expected in view of the increase in the lifestyle-related disease. In clinical point of views, one problem is progress to the hepatitis, and then, to the liver cirrhosis and liver cancer by the activation of stellate cells.

Administration of icosapentaenoic acid (hereinafter referred to as “EPA”) or fish oil to NASH or NAFLD patient has been reported. For example, amelioration of hepatitis in the patient suffering from NAFLD by administering an ω3 polyunsaturated fatty acid (hereinafter referred to as “PUFA”), and more particularly, a mixed system of ethyl icosapentate (hereinafter referred to as “EPA-E”) and ethyl docosahexaenoate (hereinafter referred to as “DHA-E”) has been proposed (see Non-Patent Literature 2). In addition, the newest report by Tanaka et al. discloses administration of 2700 mg/day of high purity EPA-E for 12 months, and amelioration of NASH was demonstrated by observing aspartate aminotransferase (hereinafter referred to as “AST”) or ALT enzyme, evaluation of inflammatory cytokine or oxidative stress marker, and liver biopsy after the administration and observation (see Non-Patent Literature 3).

“Consultation Guide of NASH and NAFLD” (Non-Patent Literature 1) by Japan Live Society describes various therapeutic methods that have been tried to improve pathological conditions of the NASH as well as their effectivity. However, this document also states that there has so far been no established therapeutic method. Exemplary drugs described include drugs for ameliorating insulin resistance such as biguanide drugs (metformin) and PPAR-γ agonist thiazolidine derivatives (pioglitazone and rosiglitazone); antioxidants such as vitamin, betaine (choline derivative), and N-acetyl cysteine; therapeutic drugs for hyperlipemia such as fibrate drugs (PPAR-α agonist), 3-hydroxy-3-methyl glutaryl coenzyme A reductase inhibitor (hereinafter referred to as “HMG-CoA reductase inhibitor”) (statin), and probucol; liver protectants such as ursodeoxycholic acid and polyenephosphatidylcholine (EPL); and angiotensin II receptor blocker (hereinafter referred to as “ARB”) such as losartan.

In addition to the literature as described above, metformin which is a biguanide hypoglycemic agent having the effect of ameliorating the insulin resistance has been described as a therapeutic agent of the NASH in view of the situation that insulin resistance is heavily involved in the mechanism of the NASH. It has been reported that oral administration of the metformin to the NASH patient at 1500 mg/day for 4 months resulted in the significant decrease in serum alanine aminotransferase (hereinafter referred to as “ALT”) level and significant increase of lactic acid level (see Non-Patent Literature 4).

The biguanide hypoglycemic agent is a compound comprising two guanidine groups bonded to each other, and this drug has been used in treating diabetes for a long time. The biguanide hypoglycemic agent is the first choice drug in the case of obese patient suffering from type II diabetes mellitus. However, gastrointestinal symptoms are often found as side effects, and more infrequent serious side effects include lactic acidosis and delayed hypoglycemia. Accordingly, biguanide hypoglycemic agents are accepted as drugs requiring cautious use.

In the meanwhile, acetylsalicylic acid (aspirin) which is a nonsteroidal anti-inflammatory drug (hereinafter referred to as “NSAID”) has been used as an antipyretic, analgesic, or a drug suppressing thrombogenesis and embolization because it has antipyretic, analgesic, anti-inflammatory, and antiplatelet actions since it inhibits prostaglandin biosynthesis. Gastrointestinal disorder, hypersensitivity, tinnitus and hearing loss by excessive administration, and the like have bee reported as the side effects of the salicylic acid drugs, and administration of the salicylic acid drugs is counterindicated for patients with the history of peptic ulcer, patients of liver disorder or patients with the history of liver disorder, and patients with the history of hypersensitivity such as aspirin-induced asthma (Non-Patent Literature 5). “Consultation Guide of NASH and NAFLD” also disclose that aspirin is responsible for steatohepatitis (microvesicular fatty liver).

HMG-CoA reductase inhibitor has been used as an anti-hyperlipemia drug since the HMG-CoA reductase inhibitor specifically and competitively inhibits HMG-CoA reductase which is a rate-limiting enzyme of the cholesterol biosynthesis system. A serious side effect of the HMG-CoA reductase inhibitor is rhabdomyolysis, and the rhabdomyolysis is most likely to occur in the patients suffering from or patients with the history of liver or renal dysfunction, and the patients who are receiving a filtrate or nicotinic acid. In view of such situation, HMG-CoA reductase inhibitor is considered an agent requiring careful use in the case of such patient. For example, muscle pain and increase of serum creatine phosphokinase (hereinafter referred to as “CPK”) may be the prodrome of rhabdomyolysis, and such prodromes need to be fully observed and the drug administration should be terminated as desired (Non-Patent Literature 6). Oral administration of pravastatin which is a HMG-CoA reductase inhibitor to NASH patient at 20 mg/day for 6 months is known to improve serum ALT level and liver tissue observation (Non-Patent Literature 7).

ARB has been used as an antihypertensive drug since the ARB mainly binds to angiotensin II receptor subtype 1 and exhibits its hypotensive action by antagonizing against angiotensin II which is a physiological pressor substance. ARB has been reported to have the risk of rapid exacerbation of kidney function in patients suffering from bilateral renal artery stenosis, and also, to have the risk of inducing shock symptoms due to transient reduction in blood pressure. Accordingly, ARB needs to be carefully administered by monitoring serum creatinine, particularly in the case of patients suffering from renal dysfunction or hepatic dysfunction (Non-Patent Literature 8). It has been known that oral administration of losartan which is an ARB for 48 weeks at 50 mg/day to a NASH patient also suffering from hypertension would invite improvement of ALT, type IV collagen, and ferritin (Non-Patent Literature 9).

With regard to the combined use of the drugs, combined use of a fibrate or a thiazolidine derivative with the ω3PUFA for treating fatty liver such as NASH has been proposed (see Patent Literature 1). This combined system has the problems such as excessive activity of PPAR-α in the combined use of the fibrate, hepatotoxicity in the combined use of the thiazolidine derivative, and exacerbation of fatty liver by PPAR-γ in basic test.

Use of the metformin as an optional ingredient in combination with a composition containing a fatty acid and an amino acid for use in prevention, delaying of the progress, or treatment of type 2 diabetes and diabetes-related diseases and conditions has been disclosed (see Patent Literature 2).

A combined use of the PUPA with the aspirin for prevention and therapy of the inflammation has been proposed while no reference is made for the hepatitis (see Patent Literature 3). Also proposed is a combination drug containing the ω3 fatty acid with the acetylsalicylic acid as a therapeutic drug for cardiovascular disease (Patent Literature 4).

As described above, combined use of the ω3PUFA with other drug for the purpose of treating the NAFLD or NASH is unknown except for the Patent Literature 1.

### CITATION LIST

### Patent Literature

Patent Literature 1: WO 2007/081773
Patent Literature 2: JP A 2006-520335
Patent Literature 3: WO 2001/060778
Patent Literature 4: EP A 1352648

### Non-Patent Literature

Non-Patent Literature 1: “Consultation Guide of NASH and NAFLD”, edited by The Japan Society of Hepatology, Bunko-do Shuppan, Aug. 22, 2006
Non-Patent Literature 2: Aliment Pharmacol Ther., Apr. 15, 2006, 23(8): 1143-51
Non-Patent Literature 3: J Clin Gastroenterol, 2008, 42(4): 413-418
Non-Patent Literature 4: Lancet, 2001, 358, 893-894
Non-Patent Literature 5: Package insert of Aspirin “Bayer”, Bayer, 2006
Non-Patent Literature 6: Package insert of Mevalotin (Registered Trademark) tablet, Daiichi Sankyo Co., Ltd, 2007
Non-Patent Literature 7: Atherosclerosis, 2004, 174: 193-196
Non-Patent Literature 8: Package insert of Nu-Lotan (Registered Trademark) tablet, Banyu Pharmaceutical Co., Ltd., 2007
Non-Patent Literature 9: Hepatology, 2004, 40(5): 1222-1225

### SUMMARY OF INVENTION

### Technical Problems

Accordingly, an object of the present invention is to provide a highly safe, high effective, and highly convenient prophylactic/ameliorating or therapeutic agent for NASH for preventing/ameliorating and treating the NAFLD, and in particular the NASH, and for suppressing the progress to the more serious liver cirrhosis/liver cancer. Another object of the present invention is to provide a method for using such agent.

### Solution to Problems

The inventors of the present invention conducted an intensive study for solving the problems as described above, and found that a combined use of an ω3PUFA with certain types drugs realizes safety and remarkable effectiveness of the level which are not observed by single administration of the corresponding agents. The present invention has been completed on the bases of such finding. Accordingly, the prophylactic/ameliorating or therapeutic agent for NAFLD and NASH provided by the present invention contains at least one member selected from the group consisting of ω3PUFAs as the first active ingredient and at least one second active ingredient selected from the group consisting of biguanide hypoglycemic agents, NSAIDs, HMG-CoA reductase inhibitor, and ARBs. More specifically, the present invention provides the following inventions.

(1) A prophylactic/ameliorating or therapeutic agent for NASH, in which at least one first ingredient selected from the group consisting of an ω3 polyunsaturated fatty acid and pharmaceutically acceptable salts and esters thereof, and at least one second ingredient selected from the group consisting of

(a) a biguanide hypoglycemic agent,

(b) an NSAID,

(c) a HMG-CoA reductase inhibitor, and

(d) an ARE.

are applied in combination as active ingredients.
(2) A prophylactic/ameliorating or therapeutic agent according to the above (1) wherein the first ingredient is at least one compound selected from EPA, docosahexaenoic acid (hereinafter referred to as “DHA”), α-linolenic acid, and pharmaceutically acceptable salts and esters thereof.
(3) A prophylactic/ameliorating or therapeutic agent according to the above (1) wherein the agent contains EPA-E and/or DHA-E as the first ingredient.
(4) A prophylactic/ameliorating or therapeutic agent according to the above (1) wherein the agent contains EPA-E as the first ingredient.
(5) A prophylactic/ameliorating or therapeutic agent according to any one of the above (1) to (4) wherein the therapeutic effect of the agent containing the combination of the first ingredient and the second ingredient is higher than the sum of the therapeutic effect of the agent solely containing the corresponding dose of the first ingredient and the therapeutic effect of the agent solely containing the corresponding dose of the second ingredient.
(6) A prophylactic/ameliorating or therapeutic agent according to any one of the above (1) to (5) wherein the agent is a composite formulation of the first ingredient and the second ingredient.
(7) A prophylactic/ameliorating or therapeutic agent according to any one of the above (1) to (5) containing the first ingredient as the active ingredient, which is for NASH of the patient who is administered with the second ingredient.
(8) A prophylactic/ameliorating or therapeutic agent according to any one of the above (1) to (5) containing the second ingredient as the active ingredient, which is for NASH of the patient who is administered with the first ingredient.
(9) A prophylactic/ameliorating or therapeutic agent according to any one of the above (1) to (7) wherein the first ingredient is applied in combination with the second ingredient by administering the first ingredient to the patient administered with the second ingredient.
(10) A prophylactic/ameliorating or therapeutic agent according to any one of the above (1) to (6) and (8) wherein the first ingredient is applied in combination with the second ingredient by administering the second ingredient to the patient administered with the first ingredient.
(11) A prophylactic/ameliorating or therapeutic agent according to any one of the above (1) to (5) wherein the agent comprises a kit of separate preparations, namely, the preparation of the first ingredient and the preparation of the second ingredient.
(12) A method for preventing/ameliorating or treating NASH comprising the step of administering the first ingredient, and the step of administering the second ingredient.
(13) A method of the above (12) wherein the two administration steps are simultaneously carried out.
(14) A method of the above (12) wherein the two administration steps are carried out at different timing.
(15) A method of the above (12) wherein at least one value selected from the group consisting of the degree of liver fibrosis determined by imaging (such as ultrasound, CT, or MRI), liver biopsy, or fibrosis marker in plasma (such as type IV collagen, hyaluronic acid, or tissue inhibitor of metalloproteinases-1 (hereinafter referred to as “TIMP-1”)); serum AST, ALT, or AST/ALT ratio; free fatty acid (hereinafter referred to as “FFA”), adiponectin, tumor necrosis factor α (hereinafter referred to as “TNFα”), high-sensitivity C-reactive protein (hereinafter referred to as “CRP”) and blood oxidative stress marker (such as ferritin or thioredoxin); insulin resistance index (homeostasis model assessment of insulin resistance, hereinafter referred to as “HOMA-IR”) is measured, and the administration is continued until this value is within the normal range.
(16) A method for relieving side effects of the second ingredient comprising the step of administering the first ingredient, and the step of administering the second ingredient.
(17) A method of the above (16) wherein the two administration steps are simultaneously carried out.
(18) A method of the above (16) wherein the two administration steps are carried out at different timing.
(19) A method of the above (16) wherein plasma lactic acid level is monitored, and when the plasma lactic acid level exceeds the normal range, at least one measure selected from the group consisting of decrease in the dose of the biguanide hypoglycemic agent; withdrawal of the administration of the biguanide hypoglycemic agent; and administration of the ω3PUFA at an increased dose is conducted until the plasma lactic acid value is within the normal range.

The first aspect of the present invention is the embodiment wherein the second ingredient is a biguanide hypoglycemic agent (a). The combined use of the ω3PUFA (first ingredient) and the biguanide hypoglycemic agent realizes safety and remarkable effectiveness of the level which are not observed by single administration of the corresponding agents. Accordingly, the prophylactic/ameliorating or therapeutic agent for NAFLD and NASH according to the first aspect of the present invention is the one containing an ω3PUFA and a biguanide hypoglycemic agent as its active ingredients. Next, the first aspect of the invention is described in further detail.

(1-1) A prophylactic/ameliorating or therapeutic agent for NASH, in which at least one member selected from the group consisting of an ω3 PUFA and pharmaceutically acceptable salts and esters thereof, and a biguanide hypoglycemic agent are applied in combination as the active ingredients.
(1-2) A prophylactic/ameliorating or therapeutic agent according to the above (1-1) wherein the ω3 PUFA or the pharmaceutically acceptable salt or ester thereof is at least one compound selected from the group consisting of EPA, DHA, α-linolenic acid, and their pharmaceutically acceptable salts and esters.
(1-3) A prophylactic/ameliorating or therapeutic agent according to the above (1-1) containing EPA-E and/or DHA-E as the ω3 PUFAs and pharmaceutically acceptable salts and esters thereof.
(1-4) A prophylactic/ameliorating or therapeutic agent according to the above (1-1) containing EPA-E as the ω3 PUFAs and pharmaceutically acceptable salts and esters thereof.
(1-5) A prophylactic/ameliorating or therapeutic agent according to any one of the above (1-1) to (1-4) wherein the biguanide hypoglycemic agent is at least one compound selected from the group consisting of metformin, buformin, phenformin, and pharmaceutically acceptable salts thereof.
(1-6) A prophylactic/ameliorating or therapeutic agent according to any one of the above (1-1) to (1-5) wherein the biguanide hypoglycemic agent is metformin hydrochloride or buformin hydrochloride.
(1-7) A prophylactic/ameliorating or therapeutic agent according to the above (1-1) wherein the ω3 PUFA or the pharmaceutically acceptable salt or ester thereof is EPA-E and/or DHA-E and the biguanide hypoglycemic agent is metformin hydrochloride or buformin hydrochloride.
(1-8) A prophylactic/ameliorating or therapeutic agent according to the above (1-1) wherein the ω3 PUFA or the pharmaceutically acceptable salt or ester thereof is EPA-E, and the biguanide hypoglycemic agent is metformin hydrochloride.
(1-9) A prophylactic/ameliorating or therapeutic agent according to any one of the above (1-1) to (1-8) wherein the therapeutic effect of the agent containing the combination of the at least one compound selected from the group consisting of ω3 PUFAs and pharmaceutically acceptable salts and esters thereof and the biguanide hypoglycemic agent is higher than the sum of the therapeutic effect of the agent solely containing the corresponding dose of the ω3 PUFA and the therapeutic effect of the agent solely containing the corresponding dose of the biguanide hypoglycemic agent.
(1-10) A prophylactic/ameliorating or therapeutic agent according to any one of the above (1-1) to (1-9) wherein the agent is a composite formulation of the at least one compound selected from the group consisting of ω3 PUFAs and pharmaceutically acceptable salts and esters thereof and the biguanide hypoglycemic agent.
(1-11) A prophylactic/ameliorating or therapeutic agent for NASH wherein the agent is the prophylactic/ameliorating or therapeutic agent according to any one of the above (1-1) to (1-9) comprising the at least one compound selected from the group consisting of ω3 PUFAs and pharmaceutically acceptable salts and esters thereof as the active ingredient, and wherein patient is administered with the biguanide hypoglycemic agent.
(1-12) A prophylactic/ameliorating or therapeutic agent for NASH wherein the agent is the prophylactic/ameliorating or therapeutic agent according to any one of the above (1-1) to (1-9) comprising the biguanide hypoglycemic agent as the active ingredient, and wherein patient is administered with the at least one compound selected from the group consisting of ω3 PUFAs and pharmaceutically acceptable salts and esters thereof.
(1-13) A prophylactic/ameliorating or therapeutic agent according to any one of the above (1-1) to (1-11) wherein the at least one compound selected from the group consisting of ω3 PUFAs and pharmaceutically acceptable salts and esters thereof is applied in combination with the biguanide hypoglycemic agent by administering the compound selected from ω3 PUFAs and pharmaceutically acceptable salts and esters thereof to the patient administered with the biguanide hypoglycemic agent.
(1-14) A prophylactic/ameliorating or therapeutic agent according to any one of the above (1-1) to (1-10) and (1-12) wherein the at least one compound selected from the group consisting of ω3 PUFAs and pharmaceutically acceptable salts and esters thereof is applied in combination with the biguanide hypoglycemic agent by administering the biguanide hypoglycemic agent to the patient administered with the at least one compound selected from the group consisting of ω3 PUFAs and pharmaceutically acceptable salts and esters thereof.
(1-15) A prophylactic/ameliorating or therapeutic agent according to any one of the above (1-1) to (1-9) wherein the agent comprises a kit of separate preparations, namely, a preparation of the at least one compound selected from the group consisting of ω3 PUFAs and pharmaceutically acceptable salts and esters thereof and a preparation of the biguanide hypoglycemic agent.
(1-16) A prophylactic/ameliorating or therapeutic agent according to any one of the above (1-1) to (1-15) wherein the combination further comprises at least one compound selected from the group consisting of liver protectant, hypoglycemic drug, antihyperlipidemia drug, antihypertensive agent, antioxidant, and anti-inflammatory agent as the active ingredient.
(1-17) A method for preventing/ameliorating or treating NASH comprising the step of administering at least one compound selected from the group consisting of ω3 PUFAs and pharmaceutically acceptable salts and esters thereof, and the step of administering a biguanide hypoglycemic agent.
(1-18) A method of the above (1-17) wherein the two administration steps are simultaneously carried out.
(1-19) A method of the above (1-17) wherein the two administration steps are carried out at different timing.
(1-20) A method of the above (1-17) wherein at least one value selected from the group consisting of improvement in the degree of liver fibrosis determined by imaging (such as ultrasound, CT, or MRI), liver biopsy, or fibrosis marker in plasma (such as type IV collagen, hyaluronic acid, or TIMP-1); serum AST, ALT, or AST/ALT ratio; FFA, adiponectin, TNFα, high-sensitivity CRP and blood oxidative stress marker (such as ferritin or thioredoxin); HOMA-IR is measured, and the administration is continued until this value is within the normal range.
(1-21) A method according to any one of the above (1-17) to (1-20) wherein the biguanide hypoglycemic agent is at least one compound selected from the group consisting of metformin, buformin, phenformin, and pharmaceutically acceptable salts thereof.
(1-22) A method for ameliorating side effects of a biguanide hypoglycemic agent comprising the step of administering at least one compound selected from the group consisting of ω3 PUFAs and pharmaceutically acceptable salts and esters thereof, and the step of administering a biguanide hypoglycemic agent.
(1-23) A method of the above (1-22) wherein the two administration steps are simultaneously carried out.
(1-24) A method of the above (1-22) wherein the two administration steps are carried out at different timing.
(1-25) A method of the above (1-22) wherein plasma lactic acid level is monitored, and when the plasma lactic acid level exceeds the normal range, at least one measure selected from the group consisting of decrease in the dose of the biguanide hypoglycemic agent; withdrawal of the administration of the biguanide hypoglycemic agent; and administration of the ω3PUFA at an increased dose is conducted until the plasma lactic acid value is within the normal range.
(1-26) A method according to any one of the above (1-22) to (1-25) wherein the biguanide hypoglycemic agent is at least one compound selected from the group consisting of metformin, buformin, phenformin, and pharmaceutically acceptable salts thereof.

The present invention provides a safe and highly effective prophylactic/ameliorating or therapeutic agent for NASH as well as a method for using such agent by combining at least one member selected from the group consisting of ω3 PUFAs and pharmaceutically acceptable salts and esters thereof with a biguanide hypoglycemic agent.

More specifically, the agent of the present invention is expected to exhibit synergetic prophylactic/ameliorative or therapeutic effects for the NASH, and in particular, synergetic prophylactic/ameliorative or therapeutic effects for the NASH including improvement of the HOMA-IR, enhancement of the adiponectin, suppression of the increase of the adipocytokine such as TNFα, and decrease of the FFA compared to the single use of the corresponding agents.

Lactic acidosis is the most serious side effect of the biguanide hypoglycemic agent, and the lactic acidosis is most likely to occur in the patients suffering from hepatic dysfunction or serious cardiovascular or lung dysfunction such as cardiac failure, myocardial infarction, or pulmonary embolism. In such patients, administration of the biguanide hypoglycemic agent is contraindicated. Administration of the biguanide hypoglycemic agent may also induce serious delayed hypoglycemia, and other major side effects which are problematic in routine practice include gastrointestinal symptoms such as anorexia, nausea, vomiting, diarrhea, constipation, and abdominal pain. In view of such situation, biguanide hypoglycemic agents are considered to be agents requiring careful use such as limitation of the dose and cautious administration. The present invention enables decrease of the dose of the drug, and in particular, dose of the biguanide hypoglycemic agents, and this enables to relieve the side effects such as lactic acidosis and delayed hypoglycemia. Decrease in the dose also enables treatment with the biguanide hypoglycemic agent of the patients who could not receive the treatment or the patients who had to stop the treatment because of the side effects of the biguanide hypoglycemic agent.

Furthermore, the present invention is capable of further improving the prophylactic/ameliorative or therapeutic effects by reducing the burden of the patients by providing the agent in the form of a composite formulation or a kit and thereby improving the drug compliance.

The second aspect of the present invention is the embodiment wherein the second ingredient is an NSAID (b). The combined use of the ω3PUFA (first ingredient) and the NSAID realizes safety and remarkable effectiveness of the level which are not observed by single administration of the corresponding agents. Accordingly, the prophylactic/ameliorating or therapeutic agent for NAFLD and NASH according to the second aspect of the present invention contains an ω3PUFA and an NSAID as its active ingredients. Next, the second aspect of the invention is described in further detail.

(2-1) A prophylactic/ameliorating or therapeutic agent for NASH, in which at least one member selected from the group consisting of an ω3 PUFA and pharmaceutically acceptable salts and esters thereof, and an NSAID are applied in combination as the active ingredients.
(2-2) A prophylactic/ameliorating or therapeutic agent according to the above (2-1) wherein the ω3 PUFA or the pharmaceutically acceptable salt or ester thereof is at least one compound selected from the group consisting of EPA, DHA, α-linolenic acid, and their pharmaceutically acceptable salts and esters.
(2-3) A prophylactic/ameliorating or therapeutic agent according to the above (2-1) containing EPA-E and/or DHA-E as the ω3 PUPA or the pharmaceutically acceptable salt or ester thereof.
(2-4) A prophylactic/ameliorating or therapeutic agent according to the above (2-1) containing EPA-E as the ω3 PUFA or the pharmaceutically acceptable salt or ester thereof.
(2-5) The prophylactic/ameliorating or therapeutic agent according to the above (2-1) wherein the NSAID is at least one member selected from acetylsalicylic acid (aspirin) and pharmaceutically acceptable salts and esters thereof.
(2-6) The prophylactic/ameliorating or therapeutic agent according to the above (2-1) wherein the ω3 PUFA or the pharmaceutically acceptable salt or ester thereof is EPA-E and/or DHA-E, and the NSAID is acetylsalicylic acid (aspirin).
(2-7) The prophylactic/ameliorating or therapeutic agent according to the above (2-1) wherein the ω3 PUFA or the pharmaceutically acceptable salt or ester thereof is EPA-E, and the NSAID is acetylsalicylic acid (aspirin).
(2-8) A prophylactic/ameliorating or therapeutic agent according to any one of the above (2-1) to (2-7) wherein the therapeutic effect of the agent containing the combination of the at least one compound selected from the group consisting of ω3 PUFAs and pharmaceutically acceptable salts and esters thereof and the at least one compound selected from acetylsalicylic acid and pharmaceutically acceptable salts and esters is higher than the sum of the therapeutic effect of the agent solely containing the corresponding dose of the ω3 PUFA and the therapeutic effect of the agent solely containing the corresponding dose of the acetylsalicylic acid or the pharmaceutically acceptable salt or ester.
(2-9) A prophylactic/ameliorating or therapeutic agent according to any one of the above (2-1) to (2-8) wherein the agent is a composite formulation of at least one compound selected from the group consisting of ω3 PUFAs and pharmaceutically acceptable salts and esters thereof and at least one compound selected from NSAIDs.
(2-10) A prophylactic/ameliorating or therapeutic agent for NASH wherein the agent is a prophylactic/ameliorating or therapeutic agent according to any one of the above (2-1) to (2-8) containing the at least one compound selected from the group consisting of ω3 PUFAs and pharmaceutically acceptable salts and esters thereof as the active ingredient, and wherein the patient is administered with the NSAID.
(2-11) A prophylactic/ameliorating or therapeutic agent for NASH wherein the agent is a prophylactic/ameliorating or therapeutic agent according to any one of the above (2-1) to (2-8) containing the at least one compound selected from NSAIDs as the active ingredient, and wherein the patient is administered with the at least one compound selected from the group consisting of ω3 PUFAs and pharmaceutically acceptable salts and esters thereof.
(2-12) A prophylactic/ameliorating or therapeutic agent according to any one of the above (2-1) to (2-10) wherein the at least one compound selected from the group consisting of ω3 PUFAs and pharmaceutically acceptable salts and esters thereof is applied in combination with the at least one compound selected from the group consisting of NSAIDs and pharmaceutically acceptable salts and esters thereof by administering the at least one compound selected from the group consisting of ω3 PUFAs and pharmaceutically acceptable salts and esters thereof to the patient administered with the NSAID.
(2-13) A prophylactic/ameliorating or therapeutic agent according to any one of the above (2-1) to (2-9) and (2-11) wherein the at least one compound selected from the group consisting of ω3 PUFAs and pharmaceutically acceptable salts and esters thereof is applied in combination with the at least one compound selected from the group consisting of NSAIDs and pharmaceutically acceptable salts and esters thereof by administering the NSAID to the patient administered with the at least one compound selected from the group consisting of ω3 PUFAs and pharmaceutically acceptable salts and esters thereof.
(2-14) A prophylactic/ameliorating or therapeutic agent according to any one of the above (2-1) to (2-8) wherein the agent comprises a kit of separate preparations, namely, a preparation of the at least one compound selected from the group consisting of ω3 PUFAs and pharmaceutically acceptable salts and esters thereof and a preparation of the at least one compound selected from the group consisting of NSAIDs and pharmaceutically acceptable salts and esters thereof.
(2-15) A prophylactic/ameliorating or therapeutic agent according to any one of the above (2-1) to (2-14) wherein the combination further comprises at least one compound selected from the group consisting of liver protectant, hypoglycemic drug, antihyperlipidemia drug, antihypertensive agent, antioxidant, and anti-inflammatory agent as the active ingredient.
(2-16) A method for preventing/ameliorating or treating NASH comprising the step of administering at least one compound selected from the group consisting of ω3 PUFAs and pharmaceutically acceptable salts and esters thereof, and the step of administering an NSAID.
(2-17) A method of the above (2-16) wherein the two administration steps are simultaneously carried out.
(2-18) A method of the above (2-16) wherein the two administration steps are carried out at different timing.
(2-19) A method of the above (2-16) wherein at least one value selected from the group consisting of improvement in the degree of liver fibrosis determined by imaging (such as ultrasound, CT, or MRI), liver biopsy, or fibrosis marker in plasma (such as type IV collagen, hyaluronic acid, or TIMP-1); serum AST, ALT, or AST/ALT ratio; FFA, adiponectin, TNFα, high-sensitivity CRP and blood oxidative stress marker (such as ferritin or thioredoxin); HOMA-IR is measured, and the administration is continued until this value is within the normal range.
(2-20) A method according to any one of the above (2-16) to (2-19) wherein the NSAID is at least one compound selected from acetylsalicylic acid and pharmaceutically acceptable salts and esters thereof.
(2-21) A method for ameliorating side effects of an NSAID comprising the steps of administering at least one compound selected from the group consisting of ω3 PUFAs and pharmaceutically acceptable salts and esters thereof, and administering an NSAID.
(2-22) A method of the above (2-21) wherein the two administration steps are simultaneously carried out.
(2-23) A method of the above (2-21) wherein the two administration steps are carried out at different timing.
(2-24) A method of the above (2-21) wherein plasma ALT level is monitored, and when the plasma ALT level exceeds the normal range, at least one measure selected from the group consisting of decrease of the dose of the NSAID; withdrawal of the administration of the NSAID; and administration of the ω3PUFA at an increased dose is conducted until the plasma ALT value is within the normal range.
(2-25) A method according to any one of the above (2-21) to (2-24) wherein the NSAID is at least one member selected from the group consisting of acetylsalicylic acid and pharmaceutically acceptable salts and esters.

The present invention provides a safe and highly effective prophylactic/ameliorating or therapeutic agent for NASH as well as a method for using such agent by combining at least one member selected from the group consisting of ω3 PUFAs and pharmaceutically acceptable salts and esters thereof with at least one member selected from the group consisting of NSAIDs, and in particular, at least one member selected from acetylsalicylic acid and pharmaceutically acceptable salts and esters.

More specifically, while single use of the acetylsalicylic acid is incapable of realizing sufficient prophylactic/ameliorative or therapeutic effects for the NASH, combined use of the ω3PUFAs and the acetylsalicylic acid is expected to exhibit superior prophylactic/ameliorative or therapeutic effects for the NASH, and in particular, synergetic prophylactic/ameliorative or therapeutic effects for the NASH including suppression of the increase of blood neutrophil count, suppression of the increase of adipocytokines such as TNFα and interleukin (hereinafter referred to as “IL”), suppression of the increase of oxidative markers such as ferritin and thioredoxin, and suppression of the increase of high sensitivity CRP compared to the single use of the ω3PUFAs.

A known side effect of the at least one compound selected from acetylsalicylic acid and pharmaceutically acceptable salts and esters is aspirin-induced asthma, and accordingly, administration of such agent is contraindicated for the patients with the history of aspirin hypersensitivity, the patients with the history of peptic ulcer, and the patients with or with the history of hepatic dysfunction. Administration of such agent may also induce tinnitus and hearing loss in the case of excessive administration, and other major side effects which are problematic in routine practice include gastrointestinal disorder, hepatic dysfunction (for example, increase of AST or ALT), and hemorrhage. In view of such situation, these drugs are considered to be those requiring careful use such as dose limitation and cautious administration. The present invention enables decrease of the dose of the drug, and in particular, dose of the at least one compound selected from acetylsalicylic acid and pharmaceutically acceptable salts and esters, and this enables to relieve the side effects such as aspirin hypersensitivity, tinnitus and hearing loss, and gastrointestinal disorder. Decrease in the dose also enables treatment with the at least one compound selected from acetylsalicylic acid and pharmaceutically acceptable salts and esters of the patients who could not receive the treatment or the patients who had to stop the treatment because of the side effects of the NSAIDs. The present invention is particularly useful in patients suffering from angina pectoris, myocardial infarction, ischemic or cerebrovascular disease, and patients who have experienced coronary artery bypass grafting or percutaneous transluminal coronary angioplasty who are in need of continued administration of the acetylsalicylic acid.

Furthermore, the present invention is capable of further improving the prophylactic/ameliorative or therapeutic effects by reducing the burden of the patients by providing the agent in the form of a composite formulation or a kit and thereby improving the drug compliance.

The third aspect of the present invention is the embodiment wherein the second ingredient is a HMG-CoA reductase inhibitor (c). The combined use of the ω3PUFA (first ingredient) and the HMG-CoA reductase inhibitor realizes safety and remarkable effectiveness of the level which are not observed by single administration of the corresponding agents. Accordingly, the prophylactic/ameliorating or therapeutic agent for NAFLD and NASH according to the third aspect of the present invention contains an ω3PUFA and a HMG-CoA reductase inhibitor as its active ingredients. Next, the third aspect of the invention is described in further detail.

(3-1) a prophylactic/ameliorating or therapeutic agent for NASH, in which at least one member selected from the group consisting of an ω3 PUFA and pharmaceutically acceptable salts and esters thereof, and a HMG-CoA reductase inhibitor are applied in combination as the active ingredients.

(3-2) A prophylactic/ameliorating or therapeutic agent according to the above (3-1) wherein the ω3 PUFA or the pharmaceutically acceptable salt or ester thereof is at least one compound selected from the group consisting of EPA, DHA, α-linolenic acid, and their pharmaceutically acceptable salts and esters.

(3-3) A prophylactic/ameliorating or therapeutic agent according to the above (3-1) containing EPA-E and/or DHA-E as the ω3 PUFA or the pharmaceutically acceptable salt or ester thereof.
(3-4) A prophylactic/ameliorating or therapeutic agent according to the above (3-1) containing EPA-E as the ω3 PUFA or the pharmaceutically acceptable salt or ester thereof.
(3-5) A prophylactic/ameliorating or therapeutic agent according to any one of the above (3-1) to (3-4) wherein the HMG-CoA reductase inhibitor is at least one compound selected from the group consisting of lovastatin, pravastatin, simvastatin, fluvastatin, atorvastatin, rosuvastatin, pitavastatin, cerivastatin, and pharmaceutically acceptable salts thereof.
(3-6) A prophylactic/ameliorating or therapeutic agent according to any one of the above (3-1) to (3-4) wherein the HMG-CoA reductase inhibitor is pravastatin sodium salt, simvastatin, fluvastatin sodium salt, atorvastatin calcium hydrate, or rosuvastatin calcium salt.
(3-7) A prophylactic/ameliorating or therapeutic agent according to the above (3-1) wherein the ω3 PUFA or the pharmaceutically acceptable salt or ester thereof is EPA-E and/or DHA-E, and the HMG-CoA reductase inhibitor is pravastatin sodium salt, simvastatin, fluvastatin sodium salt, atorvastatin calcium hydrate, or rosuvastatin calcium salt.
(3-8) A prophylactic/ameliorating or therapeutic agent according to the above (3-1) wherein the ω3 PUFA or the pharmaceutically acceptable salt or ester thereof is EPA-E, and the HMG-CoA reductase inhibitor is pravastatin sodium salt, simvastatin, fluvastatin sodium salt, atorvastatin calcium hydrate, or rosuvastatin calcium salt.
(3-9) A prophylactic/ameliorating or therapeutic agent according to any one of the above (3-1) to (3-8) wherein the therapeutic effect of the agent containing the combination of the at least one compound selected from the group consisting of ω3 PUFAs and pharmaceutically acceptable salts and esters thereof and the HMG-CoA reductase inhibitor is higher than the sum of the therapeutic effect of the agent solely containing the corresponding dose of the ω3 PUFA and the therapeutic effect of the agent solely containing the corresponding dose of the HMG-CoA reductase inhibitor.
(3-10) A prophylactic/ameliorating or therapeutic agent according to any one of the above (3-1) to (3-9) wherein the agent is a composite formulation of the at least one compound selected from the group consisting of ω3 PUFAs and pharmaceutically acceptable salts and esters thereof and the HMG-CoA reductase inhibitor.
(3-11) A prophylactic/ameliorating or therapeutic agent for NASH wherein the agent is the prophylactic/ameliorating or therapeutic agent of any one of the above (3-1) to (3-9) containing the at least one compound selected from the group consisting of ω3 PUFAs and pharmaceutically acceptable salts and esters thereof as the active ingredient and wherein the patient is administered with the HMG-CoA reductase inhibitor.
(3-12) A prophylactic/ameliorating or therapeutic agent for NASH wherein the agent is the prophylactic/ameliorating or therapeutic agent of any one of the above (3-1) to (3-9) containing the HMG-CoA reductase inhibitor as the active ingredient and wherein the patient is administered with the at least one compound selected from the group consisting of ω3 PUFAs and pharmaceutically acceptable salts and esters thereof.
(3-13) A prophylactic/alleviaive or therapeutic agent according to any one of the above (3-1) to (3-11) wherein the at least one compound selected from the group consisting of ω3 PUFAs and pharmaceutically acceptable salts and esters thereof is applied in combination with the HMG-CoA reductase inhibitor by administering the at least one compound selected from the group consisting of ω3 PUFAs and pharmaceutically acceptable salts and esters thereof to the patient administered with the HMG-CoA reductase inhibitor.
(3-14) A prophylactic/alleviaive or therapeutic agent according to any one of the above (3-1) to (3-10) and (3-12) wherein the at least one compound selected from the group consisting of ω3 PUFAs and pharmaceutically acceptable salts and esters thereof is applied in combination with the HMG-CoA reductase inhibitor by administering the HMG-CoA reductase inhibitor to the patient administered with the at least one compound selected from the group consisting of ω3 PUFAs and pharmaceutically acceptable salts and esters thereof.
(3-15) A prophylactic/ameliorating or therapeutic agent according to any one of the above (3-1) to (3-9) wherein the agent comprises a kit of separate preparations, namely, a preparation of the at least one compound selected from the group consisting of ω3 PUFAs and pharmaceutically acceptable salts and esters thereof and a preparation of the HMG-CoA reductase inhibitor.
(3-16) A prophylactic/ameliorating or therapeutic agent according to any one of the above (3-1) to (3-15) wherein the combination further comprises at least one compound selected from the group consisting of liver protectant, hypoglycemic drug, antihyperlipidemia drug, antihypertensive agent, antioxidant, and anti-inflammatory agent as the active ingredient.
(3-17) A method for preventing/ameliorating or treating NASH comprising the step of administering at least one member selected from the group consisting of ω3 PUFAs and pharmaceutically acceptable salts and esters thereof, and the step of administering an HMG-CoA reductase inhibitor.
(3-18) A method of the above (3-17) wherein the two administration steps are simultaneously carried out.
(3-19) A method of the above (3-17) wherein the two administration steps are carried out at different timing.
(3-20) A method of the above (3-17) wherein at least one value selected from the group consisting of improvement in the degree of liver fibrosis determined by imaging (such as ultrasound, CT, or MRI), liver biopsy, or fibrosis marker in plasma (such as type IV collagen, hyaluronic acid, or TIMP-1); serum AST, AZT, or AST/ALT ratio; FFA, adiponectin, TNFα, high-sensitivity CRP and blood oxidative stress marker (such as ferritin or thioredoxin); HOMA-IR is measured, and the administration is continued until this value is within the normal range.
(3-21) A method according to any one of the above (3-17) to (3-20) wherein the HMG-CoA reductase inhibitor is at least one compound selected from the group consisting of lovastatin, pravastatin, simvastatin, fluvastatin, atorvastatin, rosuvastatin, pitavastatin, cerivastatin, and a pharmaceutically acceptable salt thereof.
(3-22) A method for ameliorating side effects of an HMG-CoA reductase inhibitor comprising the steps of administering at least one compound selected from the group consisting of ω3 PUFAs and pharmaceutically acceptable salts and esters thereof, and administering an HMG-CoA reductase inhibitor.
(3-23) A method of the above (3-22) wherein the two administration steps are simultaneously carried out.
(3-24) A method of the above (3-22) wherein the two administration steps are carried out at different timing.
(3-25) A method of the above (3-22) wherein serum CPK level is monitored, and when the serum CPK level exceeds the normal range, at least one measure selected from the group consisting of decrease in the dose of the HMG-CoA reductase inhibitor at a reduced dose; withdrawal of the administration of the HMG-CoA reductase inhibitor; and administration of the ω3PUFA at an increased dose is conducted until the serum CPK level is within the normal range.

(3-26) A method according to any one of the above (3-22) to (3-25) wherein the HMG-CoA reductase inhibitor is at least one compound selected from the group consisting of lovastatin, pravastatin, simvastatin, fluvastatin, atorvastatin, rosuvastatin, pitavastatin, cerivastatin, and pharmaceutically acceptable salts thereof.

The present invention provides a safe and highly effective prophylactic/ameliorating or therapeutic agent for NASH as well as a method for using such agent by combining at least one member selected from the group consisting of ω3 PUFAs and pharmaceutically acceptable salts and esters thereof with an HMG-CoA, reductase inhibitor.

More specifically, the agent of the present invention is expected to exhibit synergetic prophylactic/ameliorative or therapeutic effects for the NASH, and in particular, synergetic prophylactic/ameliorative or therapeutic effects for the NASH including improvement in the HOMA-IR, improvement in the adipocytokine such as TNFα and IL, improvement in the high sensitivity CPR, improvement in the fibrosis marker (such as type IV collagen, hyaluronic acid, or TIMP-1), and improvement in the blood oxidative stress marker (such as ferritin and thioredoxin).

Rhabdomyolysis is a serious side effect of the HMG-CoA reductase inhibitor, and the rhabdomyolysis is most likely to occur in the patients suffering from or patients with the history of liver or renal dysfunction, and the patients who are receiving a fibrate or nicotinic acid. In view of such situation, HMG-CoA reductase inhibitor is considered an agent requiring careful use. The present invention enables decrease of the dose of various drugs, and in particular, the dose of the HMG-CoA reductase inhibitor, and this enables to relieve the side effects such as rhabdomyolysis. Decrease in the dose also enables treatment with the HMG-CoA reductase inhibitor of the patients who could not receive the treatment or the patients who had to stop the treatment because of the side effects of the HMG-CoA reductase inhibitor.

Furthermore, the present invention is capable of further improving the prophylactic/ameliorative or therapeutic effects by reducing the burden of the patients by providing the agent in the form of a composite formulation or a kit and thereby improving the drug compliance.

The fourth aspect of the present invention is the embodiment wherein the second ingredient is en ARB (d). The combined use of the ω3PUFA (first ingredient) and the ARB realizes safety and remarkable effectiveness of the level which are not observed by single administration of the corresponding agents. Accordingly, the prophylactic/ameliorating or therapeutic agent for NAFLD and NASH according to the fourth aspect of the present invention contains an ω3PUFA and an ARB as its active ingredients. Next, the fourth aspect of the invention is described in further detail.

(4-1) A prophylactic/ameliorating or therapeutic agent for NASH, in which
at least one member selected from the group consisting of an ω3 PUFA and pharmaceutically acceptable salts and esters thereof, and an ARB are applied in combination as the active ingredients.
(4-2) A prophylactic/ameliorating or therapeutic agent according to the above (4-1) wherein the ω3 PUFA or the pharmaceutically acceptable salt or ester thereof is at least one compound selected from the group consisting of EPA, DHA, α-linolenic acid, and their pharmaceutically acceptable salts and esters.
(4-3) A prophylactic/ameliorating or therapeutic agent according to the above (4-1) containing EPA-E and/or DHA-E as the ω3 PUPA or the pharmaceutically acceptable salt or ester thereof.
(4-4) A prophylactic/ameliorating or therapeutic agent according to the above (4-1) containing EPA-E as the ω3 PUFA or the pharmaceutically acceptable salt or ester thereof.
(4-5) A prophylactic/ameliorating or therapeutic agent according to any one of the (4-1) to (4-4) wherein the ARB is at least one compound selected from the group consisting of losartan, valsartan, irbesartan, eprosartan, candesartan, telmisartan, and olmesartan.
(4-6) A prophylactic/ameliorating or therapeutic agent according to the above (4-1) wherein the ARB is valsartan, irbesartan, candesartan cilexetil, or olmesartan medoxomil.
(4-7) A prophylactic/ameliorating or therapeutic agent according to the above (4-1) wherein the ω3 PUFA or the pharmaceutically acceptable salt or ester thereof is EPA-E and/or DHA-E and the ARB is valsartan, irbesartan, candesartan cilexetil, or olmesartan medoxomil.
(4-8) A prophylactic/ameliorating or therapeutic agent according to the above (4-1) wherein the ω3 PUFA or the pharmaceutically acceptable salt or ester thereof is EPA-E and the ARB is valsartan, irbesartan, candesartan cilexetil, or olmesartan medoxomil.
(4-9) A prophylactic/ameliorating or therapeutic agent according to any one of the above (4-1) to (4-9) wherein the therapeutic effect of the agent containing the combination of the at least one compound selected from the group consisting of ω3 PUFAs and pharmaceutically acceptable salts and esters thereof and the ARB is higher than the sum of the therapeutic effect of the agent solely containing the corresponding dose of the ω3 PUFA and the therapeutic effect of the agent solely containing the corresponding dose of the ARB.
(4-10) A prophylactic/ameliorating or therapeutic agent according to any one of the above (4-1) to (4-9) wherein the agent is a composite formulation of at least one compound selected from the group consisting of (ω3 PUFAs and pharmaceutically acceptable salts and esters thereof and the ARB.
(4-11) A prophylactic/ameliorating or therapeutic agent for NASH wherein the agent is a prophylactic/ameliorating or therapeutic agent of any one of the above (4-1) to (4-9) containing at least one compound selected from the group consisting of ω3 PUFAs and pharmaceutically acceptable salts and esters thereof as the active ingredient, and wherein the patient is administered with the ARB.
(4-12) A prophylactic/ameliorating or therapeutic agent for NASH wherein the agent is a prophylactic/ameliorating or therapeutic agent of any one of the above (4-1) to (4-9) containing the ARB as its active ingredient, and wherein the patient is administered with at least one member selected from the group consisting of ω3 PUFAs and pharmaceutically acceptable salts and esters thereof.
(4-13) A prophylactic/ameliorating or therapeutic agent according to any one of the above (4-1) to (4-11) wherein the at least one compound selected from the group consisting of ω3 PUFAs and pharmaceutically acceptable salts and esters thereof is applied in combination with the ARB by administering the ARB to the patient administered with the at least one compound selected from the group consisting of ω3 PUFAs and pharmaceutically acceptable salts and esters thereof.
(4-14) A prophylactic/ameliorating or therapeutic agent according to any one of the above (4-1) to (4-10) and (4-12) wherein the at least one compound selected from the group consisting of ω3 PUFAs and pharmaceutically acceptable salts and esters thereof is applied in combination with the ARB by administering the at least one compound selected from the group consisting of ω3 PUFAs and pharmaceutically acceptable salts and esters thereof to the patient administered with the ARB.
(4-15) A prophylactic/ameliorating or therapeutic agent according to any one of the above (4-1) and (4-9) wherein the agent is a kit of separate preparations, namely, a preparation of at least one compound selected from the group consisting of ω3 PUFAs and a preparation of pharmaceutically acceptable salts and esters thereof and an ARB.
(4-16) A prophylactic/ameliorating or therapeutic agent according to any one of the above (4-1) to (4-15) wherein the combination further comprises at least one compound selected from the group consisting of liver protectant, hypoglycemic drug, antihyperlipidemia drug, antihypertensive agent, antioxidant, and anti-inflammatory agent as the active ingredient.
(4-17) A method for preventing/ameliorating or treating NASH comprising the step of administering at least one compound selected from the group consisting of ω3 PUFAs and pharmaceutically acceptable salts and esters thereof, and the step of administering an ARB.
(4-18) A method of the above (4-17) wherein the two administration steps are simultaneously carried out.
(4-19) A method of the above (4-17) wherein the two administration steps are carried out at different timing.
(4-20) A method of the above (4-17) wherein at least one value selected from the group consisting of improvement in the degree of liver fibrosis determined by imaging (such as ultrasound, CT, or MRI), liver biopsy, or fibrosis marker in plasma (such as type TV collagen, hyaluronic acid, or TIMP-1); serum AST, ALT, or AST/ALT ratio; FFA, adiponectin, TNFα, high-sensitivity CRP and blood oxidative stress marker (such as ferritin or thioredoxin); HOMA-IR is measured, and the administration is continued until this value is within the normal range.
(4-21) The method according to any one of the above (4-17) to (4-20) ARB wherein the ARB is at least one compound selected from the group consisting of losartan, valsartan, irbesartan, eprosartan, candesartan, telmisartan, olmesartan, and pharmaceutically acceptable salts thereof.
(4-22) A method for ameliorating side effects of an ARB comprising the steps of administering at least one compound selected from the group consisting of ω3 PUFAs and pharmaceutically acceptable salts and esters thereof, and administering an ARB.
(4-23) A method of the above (4-22) wherein the two administration steps are simultaneously carried out.
(4-24) A method of the above (4-22) wherein the two administration steps are carried out at different timing.
(4-25) A method of the above (4-22) wherein serum creatinine level is monitored, and when the serum creatinine level exceeds the normal range, at least one measure selected from the group consisting of decrease in the dose of the ARB; withdrawal of the administration of the ARB; and administration of the ω3PUFA at an increased dose is conducted until the serum creatinine level is within the normal range.
(4-26) The method according to any one of the above (4-22) to (4-25) ARB wherein the ARB is at least one compound selected from the group consisting of losartan, valsartan, irbesartan, eprosartan, candesartan, telmisartan, olmesartan, and pharmaceutically acceptable salts thereof.

The present invention provides a safe and highly effective prophylactic/ameliorating or therapeutic agent for NASH as well as a method for using such agent by combining at least one member selected from the group consisting of ω3 PUFAs and pharmaceutically acceptable salts and esters thereof with an ARB.

More specifically, the agent of the present invention is expected to exhibit synergetic prophylactic/ameliorative or therapeutic effects for the NASH, and in particular, synergetic prophylactic/ameliorative or therapeutic effects for the NASH including improvement in the HOMA-IR and the fibrosis marker (such as type IV collagen, hyaluronic acid, and TIMP-1) in NASH patients also suffering from hypertension or metabolic syndrome compared to the single use of the corresponding agents.

ARB is reported to be associated with the risk of rapid aggravation of the renal function in the patients suffering from bilateral renal artery stenosis, and also, with the risk of shock symptoms by transient hypotension. In view of such situation, ARB is considered an agent requiring careful use particularly for the patients suffering from renal dysfunction or hepatic dysfunction. The present invention enables decrease of the dose of various drugs, and in particular, the dose of the ARB, and this enables to relieve the side effects such as renal dysfunction, hepatic dysfunction, or shock symptoms by transient hypotension. Decrease in the dose also enables treatment with the ARB of the patients who could not receive the treatment or the patients who had to stop the treatment because of the side effects of the ARB.

Furthermore, the present invention is capable of further improving the prophylactic/ameliorative or therapeutic effects by reducing the burden of the patients by providing the agent in the form of a composite formulation or a kit and thereby improving the drug compliance.

### ADVANTAGEOUS EFFECTS OF INVENTION

The prophylactic/ameliorating or therapeutic agent for NASH, in which at least one first ingredient selected from the group consisting of an ω3 polyunsaturated fatty acid and pharmaceutically acceptable salts and esters thereof and at least one second ingredient selected from the group consisting of a biguanide hypoglycemic agent (a), an NSAID (b), an HMG-CoA reductase inhibitor (c), and an ARB (d) are applied in combination as the active ingredients is expected to have a synergetic prophylactic/ameliorative or therapeutic effects for the NASH compared to the single use of the corresponding agents. The present invention is also capable of reducing the close compared to the single use of the corresponding active ingredients. Furthermore, the present invention is capable of further improving the prophylactic/ameliorative or therapeutic effects by reducing the burden of the patients by providing the agent in the form of a composite formulation or a kit and thereby improving the drug compliance.

### DESCRIPTION OF EMBODIMENTS

Next, the present invention is described in detail.

The present invention provides a prophylactic/ameliorating or therapeutic agent for NASH, in which at least one member selected from the group consisting of an ω3 polyunsaturated fatty acid (first ingredient) and at least one member selected from the group consisting of biguanide hypoglycemic agents, NSAIDs, HMG-CoA reductase inhibitors, and ARBs (second ingredient) are applied in combination as the active ingredients. The present invention also provides a method for using such agent. In other words, the prophylactic/ameliorating or therapeutic agent of the present invention provides a combination drug to be applied in combination of the first and the second ingredients which are the active ingredients as well as the method for using such combination drug.

The term “prophylactic” or “prevention” used in the present invention includes not only the prevention of the onset of the disease but also delaying of the onset and reduction of the incidence rate of the disease.

The term “amelioration” or “improvement” used in the present invention includes not only the amelioration of some parameter of the disease, but also, amelioration of subjective symptoms and QOL (quality of life) of the patient. The term “therapy” used in the present invention includes not only the administration of the drug to the patient who has already developed the disease, but also the administration of the drug to the patient who has high risk of developing the disease as a prophylactic treatment.

### <First Ingredient>

polyunsaturated fatty acids (PUFAs) are defined to be a fatty acid having a plurality of carbon-carbon double bonds in the molecule, and PUFAs are classified into ω3, ω6, and the like depending on the position of the double bond. Exemplary ω3PUFAs include α-linolenic acid, EPA, and DHA.

The term “PUFAs” used in the present invention includes not only the polyunsaturated fatty acids but also derivatives of the polyunsaturated fatty acids such as pharmaceutically acceptable salts, esters, amides, phospholipids, and glycerides thereof.

The ω3PUFA used in the present invention may be either synthetic, semi-synthetic, or natural ω3PUFA, or alternatively a natural oil containing any of such ω3PUFA. The natural ω3PUFAs includes the one extracted from a natural oil containing the ω3PUFA, the one partially purified from such oil, or the one further purified to a higher degree from such partially purified product. The semi-synthetic ω3PUFAs include a polyunsaturated fatty acid produced by a microorganism or the like, such polyunsaturated fatty acid or other natural polyunsaturated fatty acid which has been chemically treated, for example, by esterification, ester exchange reaction, or the like. In the present invention, the ω3PUFA used may be any of these used alone or in combination of two or more.

Exemplary ω3PUFAs used in the present invention include EPA, DHA, α-linolenic acid, and pharmaceutically acceptable salts and esters thereof. Exemplary pharmaceutically acceptable salts and esters include salts of an inorganic base such as sodium salt and potassium salt; salts of an organic base such as benzylamine salt and diethylamine salt; salts with a basic amino acid such as arginine salt and lysine salt; and alkyl esters such as ethyl ester; and esters such as mono-, di-, and triglycerides. The preferred is the ethyl ester, and the most preferred is EPA-E and/or DHA-E.

The ω3PUFA is not particularly limited for its purity. However, content of the ω3PUFA in the entire fatty acid present in the composition of the agent of the present invention is preferably at least 25% by weight, more preferably at least 50% by weight, still more preferably at least 70% by weight, and still more preferably at least 85% by weight, and the most preferred is the embodiment in which the composition of the agent of the present invention does not essentially contain any fatty acid ingredient other than the ω3PUFA. For example, when EPA-F and DHA-E are used, compositional ratio of the EPA-E/DHA-E and content of the EPA-E+DHA-E in the entire fatty acid are not particularly limited, while the compositional ratio EPA-E/DHA-E is preferably at least 0.8, more preferably 1.0, and still more preferably, at least 1.2. The EPA-E+DHA-E is preferably the one having a high purity, for example, the one having a content ratio of at least 40% by weight, more preferably at least 55% by weight, still more preferably at least 84% by weight, and even more preferably at least 96.5% by weight in the entire fatty acid and the derivatives thereof. Preferably, the agent of the present invention may have the lowest possible content of other long chain saturated fatty acids, and lowest possible content of the long chain unsaturated fatty acid, and in particular, ω6 long chain unsaturated fatty acid, and especially arachidonic acid. Content of other long chain saturated fatty acids is preferably less than 2% by mass, and more preferably less than 1% by weight.

Compared to fish oil or concentrated fish oil, the EPA-E and/or DHA-E used in the prophylactic/ameliorating or therapeutic agent of the present invention has lower content of impurities which are unfavorable for cardiovascular events such as saturated fatty acids or arachidonic acid, and therefore, intended merits can be realized without causing problems such as overnutrition or excessive intake of vitamin A. The EPA-E and/or DHA-E used in the prophylactic/ameliorating or therapeutic agent of the present invention which is an ester has higher stability to oxidation compared to fish oils which are triglycerides, and a composition having a sufficient stability can be prepared in the present invention by adding an antioxidant commonly used in the art.

The EPA-E used in the present invention may be a high purity EPA-E soft capsule for treating arteriosclerosis obliterans (ASO) and hyperlipemia which is commercially available in Japan and having a purity of at least 96.5% by weight (having a product name of Epadel manufactured by Mochida Pharmaceutical Co.). The mixture of the EPA-E and DHA-E used may be, for example, Lovaza (a soft capsule of GlaxoSmithKline containing about 46.5% by weight of EPA-E and about 37.5% by weight of DHA-E) which is a commercially available drug for treating hypertriglyceridaemia.

The ω3PUFA used may also be a purified fish oil, and use of monoglyceride, diglyceride, tritriglyceride of the ω3PUFAs, and combinations thereof also constitutes preferable embodiments. Examples of commercially available products containing the ω3PUFA or a salt or an ester thereof include Incromega F2250, F2628, E2251, F2573, TG2162, TG2779, TG2928, TG3525, and E5015 (Croda International PLC, Yorkshire, England) and EPAX6000FA, EFAX5000TG, EPAX4510TG, EPAX2050TG, EPAX7010EE, K85TG, K85EE, and K80EE (Pronova Biopharma, Lysaker, Norway), and such product may be used in the present invention.

### <Second Ingredient>

### (a) Biguanide Hypoglycemic Agent

Exemplary biguanide hypoglycemic agents used in the present invention include metformin, buformin, phenformin, and pharmaceutically acceptable salts thereof. The preferred is the use of metformin hydrochloride or buformin hydrochloride, and the most preferred is the use of metformin hydrochloride. In the present invention, the term “biguanide hypoglycemic agent” also includes such salts unless otherwise noted.

Of these, metformin hydrochloride is commercially available in Japan as Melbin (Registered Trademark) tablet (Dainippon Sumitomo Pharma Co., Ltd.), and buformin hydrochloride is available in Japan as Dibetos S tablet (Zeria Pharmaceutical Co., Ltd.). Extended release tablets such as Glucophage XR (Registered Trademark) (Bristol Mayer Squibb) and Fortamet (Registered Trademark) (Andrx Labs) are also commercially available in the U.S. These may also be used in the present invention. In view of reducing the side effect, use of such extended release tablet is preferable.

In the agent of the present invention containing the ω3PUFAs in combination with the biguanide hypoglycemic agent as described above, a preferable embodiment is the combination of EPA-E and/or DHA-E with metformin hydrochloride and/or buformin hydrochloride, and the most preferable embodiment is the combination of EPA-E and/or DHA-E with metformin hydrochloride.

### (b) Nonsteroidal Anti-Inflammatory Agent (NSAIDs)

Examples of the NSAID used in the present invention include salicylic acid derivatives such as acetylsalicylic acid and salicylic acid; indomethacin, diclofenac, ibuprofen, ketoprofen, naproxen, and piroxicam, and the preferred are acetylsalicylic acid and pharmaceutically acceptable salts and esters thereof. Examples of the pharmaceutically acceptable salt or ester of the acetylsalicylic acid include salts of an inorganic base such as sodium salt and potassium salt; salts of an organic base such as benzylamine salt and diethylamine salt; salts with a basic amino acid such as arginine salt and lysine salt; and alkyl, esters such as ethyl ester; and acetylsalicylsalicylic acid which is an ester with salicylic acid. In the present invention, the term “acetylsalicylic acid” also includes such salts unless otherwise noted. The preferred is acetylsalicylic acid.

Of these, acetylsalicylic acid is commercially available in Japan, for example, as Aspirin “Bayer”, Bayaspirin (Registered Trademark) tablet (Bayer) which is an enteric-coated tablet coated with an acid resistant film, or Bufferin 81 mg tablet (Zion Corporation) having an antacid such as Dialuminate (aluminum glycinate and magnesium carbonate) added thereto. Extended release tablets such as 8-Hour Bayer (Bayer and Measurin (Bayer) are also commercially available in the U.S. These may also be used in the present invention. In view of reducing the side effect, use of such enteric-coated tablet, preparation having an antacid added thereto, or extended release tablet is more preferable. In addition, the present invention is expected to exhibit the intended effects, for example, antiplatelet action at a dose lower than the close required to exhibit anti-inflammatory or analgesic action, and in such a case, use of Bayaspirin (Registered Trademark) tablet (100 mg) or Bufferin tablet (81 mg) is preferable.

In the agent of the present invention containing the ω3PUFAs in combination with the acetylsalicylic acid as described above, a preferable embodiment is the combination of EPA-E and/or DHA-E with acetylsalicylic acid, and the most preferable embodiment is the combination of EPA-E with acetylsalicylic acid.

### (c) HMG-CoA Reductase Inhibitor

Examples of the HMG-CoA reductase inhibitor used in the present invention include lovastatin, pravastatin, simvastatin, fluvastatin, atorvastatin, rosuvastatin, pitavastatin, cerivastatin, LCP-AtorFen (LifeCycle Pharma), RBx-10558 (Ranbaxy Laboratories), Carvastatin (Tobishi Pharmaceutical Co., Ltd., NCX-6560 (NicOx), and pharmaceutically acceptable salts and esters thereof. Examples of the preferable HMG-CoA reductase inhibitors include lovastatin, pravastatin sodium salt, simvastatin, fluvastatin sodium salt, atorvastatin calcium hydrate, rosuvastatin calcium salt, pitavastatin calcium salt, and cerivastatin sodium salt, and the more preferable examples include pravastatin sodium salt, simvastatin, fluvastatin sodium salt, atorvastatin calcium hydrate, and rosuvastatin calcium salt. In the present invention, the term “HMG-CoA reductase inhibitor” also includes such salts unless otherwise noted.

Of these, lovastatin is commercially available as Mevacor (Registered Trademark) tablet (Merck), pravastatin sodium salt is commercially available as Mevalotin (Registered Trademark) tablet (Daiichi Sankyo Co., Ltd.), simvastatin is commercially available as Lipovas (Registered Trademark) tablet (Banyu Pharmaceutical Co., Ltd.), fluvastatin sodium salt is commercially available as Lochol (Registered Trademark) tablet (Novartis Pharma K.K.), atorvastatin calcium hydrate is commercially available as Lipitor (Registered Trademark) tablet (Astellas Pharma Inc.), rosuvastatin calcium salt is commercially available as Crestor (Registered Trademark) tablet (AstraZeneca K.K.), and pitavastatin calcium salt is commercially available as Livalo (Registered Trademark) tablet (Kowa Company, Ltd.), and these may be used in the present invention. In view of reducing the side effect, use of Lescol (Registered Trademark) XL tablet (Novartis) which is an extended release tablet of the fluvastatin sodium salt commercially available in the U.S. is also preferable. Also, composite formulations with an extended release tablet of niacin which is a drug for hyperlipemia are commercially available as Advicor (Registered Trademark) tablet (Kos Pharmaceuticals) and Simcor (Registered Trademark) tablet (Abbott); a composite formulation with ezetimibe is commercially available as Vytrin (Registered Trademark) tablet (Merck/Schering-Plough Pharmaceuticals); and a composite formulation with amlodipine which is a calcium antagonist is commercially available as Caduet (Registered Trademark) tablet (Pfizer Labs), and use of these drugs may be preferable in the case of NASH patients also suffering from serious hyperlipemia or hypertension.

In the agent of the present invention containing the ω3PUFAs in combination with the HMG-CoA reductase inhibitor as described above, a preferable embodiment is the combination of EPA-E and/or DHA-E with at least one compound selected from the group consisting of lovastatin, pravastatin sodium salt, simvastatin, fluvastatin sodium salt, atorvastatin calcium hydrate, rosuvastatin calcium salt, and pitavastatin calcium salt, and the most preferable embodiment is the combination of EPA-E and/or DHA-E with at least one compound selected from the group consisting of pravastatin sodium salt, simvastatin, fluvastatin sodium salt, atorvastatin calcium hydrate, and rosuvastatin calcium salt.

### (d) Angiotensin II Receptor Blocker (ARB)

Examples of the ARB used in the present invention include losartan, valsartan, irbesartan, eprosartan, candesartan, telmisartan, olmesartan, Allisartan (Allist Pharmaceutical), Azilsartan, TAK-491 and TAK-591 (manufactured by Takeda Pharmaceutical Co., Ltd.), CYT-006-AngQb (Cytos Biotechnology), PS-433540 (Bristol-Myers Squibb Pharmaceutical), VNP-489 (Novartis), CR-3834 (Rottapharm), EK-112 (National Taiwan University), LY-285434 (Eli Lilly), Saprisartan (Glaxo Group Research) and their pharmaceutically acceptable salts and esters. The ARB used in the present invention is preferably losartan potassium salt, valsartan, irbesartan, eprosartan mesylate, candesartan cilexetil, telmisartan, or olmesartan medoxomil, and more preferably valsartan, irbesartan, candesartan cilexetil, or olmesartan medoxomil. In the present invention, the ARB also includes such salts unless otherwise noted.

Losartan potassium salt is commercially available as Nu-Lotan (Registered Trademark) tablet (Banyu Pharmaceutical Co., Ltd.); valsartan is commercially available as Diovan (Registered Trademark) tablet (Novartis Pharma K.K.); irbesartan is commercially available as Irbetan (Registered Trademark) tablet (Shionogi & Co., Ltd.) or Avapro (Registered Trademark) tablet (Sanofi Synthelabo); eprosartan mesylate is commercially available as Teveten (Registered Trademark) tablet (Smithkline Beecham Pharmaceuticals); candesartan cilexetil is commercially available as Blopress (Registered Trademark) tablet (Takeda Pharmaceutical Co., Ltd.); telmisartan is commercially available as Micardis (Registered Trademark) tablet (Boehringer Ingelheim Japan); and olmesartan medoxomil is commercially available as Olmetec (Registered Trademark) tablet (Daiichi Sankyo Co., Ltd.), and these commercially available or will-be-available products may be used in the present invention. Also, the ARB is combined with hydrochlorothiazide (a diuretic drug) and commercially available such composite formulations include Atacand (Registered Trademark) HCT (Astrazeneca), Avalide (Registered Trademark) (Sanifi Synthelabo), Benicar (Registered Trademark) HCT (Daiichi Sankyo), Diovan (Registered Trademark) HCT (Novartis), Hyzzar (Registered Trademark) (Merck), Micardis (Registered Trademark) HCT (Boehringer Ingelheim), Teveten (Registered Trademark) HCT (Smithkline Beecham Pharmaceuticals). Furthermore, the ARB is also combined with amlodipine (calcium antagonist), and commercially available such composite formulations include Azor (Registered Trademark) (Dajichi Sankyo) and Exforge (Registered Trademark) (Novartis). Use of such commercially available product is also preferable in the case of NASH patient also suffering from hypertension.

In the agent of the present invention containing the ω3PUFAs in combination with the ARB as described above, a preferable embodiment is the combination of EPA-E and/or DHA-E with at least one compound selected from the group consisting of losartan potassium salt, valsartan, irbesartan, eprosartan mesylate, candesartan cilexetil, telmisartan, and olmesartan medoxomil, and the most preferable embodiment is the combination of EPA-E and/or DHA-E with at least one compound selected from the group consisting of valsartan, irbesartan, candesartan cilexetil, and olmesartan medoxomil.

### <Embodiments of the Combined Use>

In the present invention, “combined use” of the active ingredients means use of the active ingredients in combination, and it includes both the administration of the first and the second ingredients as ingredients in the composite formulation containing the first and the second ingredients, and the administration of the first and the second ingredients as separate preparations at the same timing or at different timing with time lag. The embodiment of “the administration as separate preparations at the same timing or at different timing with time lag” include both (1) the embodiment in which the patient receiving the first ingredient is administered with a composition containing the second ingredient as its active ingredient, and (2) the embodiment in which the patient receiving the second ingredient is administered with a composition containing the first ingredient as its active ingredient. The “combined use” may not necessarily mean that both drugs are simultaneously present in the patient's body, for example, in the patient's blood, and the term “combined use” used in the present invention designates the embodiment in which the drug is administered when the effect and/or action of the other drug is still being developed in the body of the patient, namely, the embodiment which realizes the prophylactic/ameliorative or therapeutic effects of the diseases associated with NAFLD or NASH by using the prophylactic/ameliorating or therapeutic agent of the present invention. The preferred is the embodiment in which both drugs are simultaneously present in the patient's body, for example, in the patients' blood, and also preferred is the embodiment in which the other drug is administered within 24 hours after the administration of the first drug.

The embodiment of the “combined use” of the prophylactic/ameliorating or therapeutic agent of the present invention is not particularly limited as long as the active ingredients are used in combination. Exemplary such embodiments of the drug administration include, for example, (1) administration of single preparation having both active ingredients incorporated therein; (2) administration of both active ingredients by preparing separate preparations each containing different active ingredients, and simultaneously administering these separate preparations from the same administration route with or without producing a kit of the combination of two preparations; (3) administration of both active ingredients by preparing separate preparations each containing different active ingredients, and administering these separate preparations at different timing with time lag from the same administration route with or without producing a kit of the combination of two preparations; (4) administration of both active ingredients by preparing separate preparations each containing different active ingredients, and simultaneously administering these separate preparations from different administration routes (of the same patient from different site) with or without producing a kit of the combination of two preparations; and (5) administration of both active ingredients by preparing separate preparations each containing different active ingredients, and administering these separate preparations at different timing with time lag from different administration routes (of the same patient from different site) with or without producing a kit of the combination of two preparations.

When the active ingredients are administered at different timing with time lag, the first and the second ingredients may be administered in this order, or in opposite order. When the active ingredients are administered simultaneously, these ingredients may be mixed immediately before the administration if the administration route is the same, while the active ingredients may be separately administered. The active ingredients may be used deliberately at different timing for various purposes. In an exemplary embodiment, one ingredient may be administered, and thereafter, the other ingredient may be administered while the effect of the first ingredient is about to be developed or the effect of the first ingredient is still fully developed.

In another embodiment, one drug, and in particular, the second ingredient may be administered once or twice a day by using an extended release formulation, and the other ingredient, and in particular, the first ingredient may be administered two or more times, for example, twice or three times a day, or alternatively, once or twice a day by using an extended release formulation. When both drugs are administered once or twice a day, and more preferably, when both drugs are administered once or twice a day simultaneously, or administered by incorporating in a composite formulation, the burden of the patients can be reduced to improve the drug compliance, and in turn, to improve the prophylactic/ameliorative or therapeutic effects and reduce the side effect. It is also possible that both drugs are administered and one drug is withdrawn while the effects of the ingredients are about to be developed or the effects of the ingredients are still fully developed.

When the drug administration is withdrawn, close of the drug may be reduced in stepwise manner. It is also possible that one drug is administered during the withdrawal period of the other drug.

The embodiments of the use of the prophylactic/ameliorating or therapeutic agent for NASH of the present invention are not particularly limited as long as it is used in an embodiment wherein the therapeutic effects of the active ingredients, namely, at least the first and the second ingredients are realized. Exemplary such embodiments include the embodiment wherein only the first and the second ingredients are used, namely, the prophylactic/ameliorating or therapeutic agent for NASH comprising the combination of the first ingredient and the second ingredient, and the embodiment of the prophylactic/ameliorating or therapeutic agent for NASH further comprising an additional active ingredient.

Preferred embodiments are those in which the therapeutic effect realized by the combined use of the first ingredient and the second ingredient is higher than the sum of the therapeutic effect that would have been achieved when the first and second ingredients were separately used at the same dose. The term “therapeutic effect” is not particularly limited as long as it is the prophylactic/ameliorative or therapeutic effects for the disease related to NAFLD or NASH or suppressive effects for the liver cirrhosis or liver cancer. Exemplary therapeutic effects in the second aspect of the invention wherein the second ingredient is biguanide hypoglycemic agent include degree of liver fibrosis determined by imaging (such as ultrasound, CT, or MRI), liver biopsy, or a fibrosis marker in plasma (such as type IV collagen, hyaluronic acid, or TIMP-1), decrease in serum AST or ALT level, decrease in AST/ALT ratio, increase in adiponectin, decrease in TNFα, decrease in high sensitivity CPR, decrease in FFA, decrease in a blood oxidative stress marker (such as ferritin or thioredoxin), and improvement in HOMA-IR, and the preferred are increase in adiponectin, decrease in TNFα, and improvement in HOMA-IR.

Exemplary therapeutic effects in the second aspect of the invention include, for example, degree of liver fibrosis determined by the imaging, liver biopsy, or the fibrosis marker in plasma, decrease in the serum AST or ALT, decrease in the AST/ALT ratio, increase in the adiponectin, decrease in the TNFα or IL, decrease in the high sensitivity CPR, decrease in the blood oxidative stress marker (such as ferritin or thioredoxin), and decrease in the neutrophil count, and the preferred are decrease in the TNFα, IL, or high sensitivity CPR, decrease in the blood oxidative stress marker (such as ferritin or thioredoxin), and decrease in the neutrophil count.

Exemplary therapeutic effects in the third aspect of the invention include, for example, degree of liver fibrosis determined by the imaging, liver biopsy, or the fibrosis marker in plasma, decrease in the serum AST or ALT, decrease in the AST/ALT ratio, increase in the adiponectin, decrease in the TNFα or IL, decrease in the high sensitivity CPR, decrease in the blood oxidative stress marker (such as ferritin or thioredoxin), and decrease in the neutrophil count, and the preferred are improvement in the HOMA-IR, decrease in the TNFα, IL, or high sensitivity CPR, and decrease in the fibrosis marker (such as type IV collagen, hyaluronic acid, or TIMP-1) and blood oxidative stress marker (such as ferritin or thioredoxin).

Exemplary therapeutic effects in the fourth aspect of the invention include, for example, degree of liver fibrosis determined by the imaging, liver biopsy, or the fibrosis marker in plasma, decrease in the serum AST or ALT, decrease in the AST/ALT ratio, increase in the adiponectin, decrease in the TNFα, decrease in the high sensitivity CPR, decrease in the blood oxidative stress marker (such as ferritin or thioredoxin), and decrease in the neutrophil count, the preferred are decrease in the fibrosis marker (such as type IV collagen, hyaluronic acid, and TIMP-1) and improvement in the HOMA-IR.

The prophylactic/ameliorative or therapeutic effects may be monitored by using biochemical, pathological, or pathologic parameter related to the NAFLD or NASH.

The dose and dosage period of the first ingredient and the second ingredient used in the prophylactic/ameliorating or therapeutic agent of the present invention may be the dose and the period sufficient for the development of the intended action. Such dose and dosage period may be adequately adjusted depending on the dosage form, administration route, number of administration per day, seriousness of the symptom, body weight, age, and the like.

When orally administered, the first ingredient, for example, EPA-E and/or DHA-E may be administered at 0.1 to 10 g/day, preferably 0.3 to 6 g/day, preferably 0.6 to 4 g/day, and more preferably 0.9 to 2.7 g/day in one to three doses, or if desired the entire dose may be administered in one to several doses. The dose may also be reduced depending on the dose of the second ingredient. The administration is preferably conducted during or after the meal, and more preferably, immediately after the meal (within 30 minutes after the meal). In view of reducing the side effect, preferred is the use of minimized daily dose and administration of once a day by using an extended release tablet. When such dose is orally administered, the administration period is at least 1 year, preferably at least 2 years, more preferably at least 3.5 years, still more preferably at least 5 years. However, the administration is continued as long as pathological conditions or biochemical index related to NASH or high risk of onset and/or recurrence of the NASH is recognized. Possible embodiments also include administration on every alternate day, administration of 2 to 3 days per week, and inclusion of a washout period of about 1 day to 3 months, and preferably 1 week to 1 month.

The dose of the second ingredient used in the prophylactic/ameliorating or therapeutic agent of the present invention is preferably within the dose and administration route when the drug of the second ingredient is used alone. Such dose, however, may be adequately adjusted depending on the type, dosage form, administration route, number of the administration per day, seriousness of the symptom, body weight, age, and the like.

When the second ingredient is orally administered in the first aspect of the present invention, for example, metformin hydrochloride may be administered at 10 to 5000 mg/day, preferably at 100 to 1000 mg/day, and more preferably at 500 to 750 mg/day in one to three doses, or if desired, in several doses per day. Depending on the physician's indication, a daily dose lower than the physician's recommendation (for example, 10 to 300 mg) may be orally administered on the first day of the administration, and thereafter, a dose gradually increasing to the daily maximum dose (of up to 2550 mg) may be orally administered as the maintenance dose. The dose of the second ingredient may also be reduced depending on the dose of the first ingredient. In view of reducing the side effect, preferred is the use of minimized daily dose and administration of once a day by using an extended release tablet. When such dose is orally administered, the administration period is at least 1 year, preferably at least 2 years, more preferably at least 3.5 years, still more preferably at least 5 years. However, the administration is continued as long as pathological conditions or biochemical index related to NASH or high risk of onset and/or recurrence of the NASH is recognized. Possible embodiments also include administration on every alternate day, administration of 2 to 3 days per week, and inclusion of a washout period of about 1 day to 3 months, and preferably 1 week to 1 month.

In the second aspect of the present invention, the administration of the second ingredient is preferably conducted after the meal, and interdigestive administration is not preferable. When the second ingredient is orally administered, for example, the second ingredient may be administered at 50 to 4500 mg/day, preferably at 100 to 2000 mg/day, and more preferably at 300 to 1500 mg/day in one to three doses, or if desired in several doses per day. Depending on the physician's indication, a daily dose higher than the physician's recommendation (for example, a dose several times higher than the physician's recommendation) may be orally administered on the first day of the administration, and thereafter, the daily maximum dose may be orally administered as the maintenance dose. The dose of the second ingredient may also be reduced depending on the dose of the first ingredient. In view of reducing the side effect, preferred is the use of minimized daily dose and administration of once or twice a day by using an enteric-coated tablet, a tablet having an antiacid added thereto, or an extended release tablet. When such dose is orally administered, the administration period is at least 1 year, preferably at least 2 years, more preferably at least 3.5 years, still more preferably at least 5 years. However, the administration is continued as long as pathological conditions or biochemical index related to NASH or high risk of onset and/or recurrence of the NASH is recognized. Possible embodiments also include administration on every alternate day, administration of 2 to 3 days per week, and inclusion of a washout period of about 1 day to 3 months, and preferably 1 week to 1 month.

When the second ingredient is orally administered in the third aspect of the present invention, for example, lovastatin may be administered at 0.5 to 1000 mg/day, preferably at 5 to 500 mg/day, and more preferably at 10 to 80 mg/day; pravastatin sodium salt may be administered at 0.5 to 1000 mg/day, preferably at 5 to 500 mg/day, and more preferably at 10 to 80 mg/day; simvastatin may be administered at 0.2 to 1000 mg/day, preferably at 2 to 500 mg/day, and more preferably at 5 to 80 mg/day; fluvastatin sodium salt may be administered at 1 to 500 mg/day, preferably at 10 to 200 mg/day, and more preferably at 20 to 80 mg/day; atorvastatin calcium hydrate may be administered at 0.5 to 500 mg/day, preferably at 5 to 200 mg/day, and more preferably at 10 to 80 mg/day; rosuvastatin calcium salt may be administered at 0.1 to 100 mg/day, preferably at 1 to 60 mg/day, and more preferably at 2.5 to 40 mg/day; and pitavastatin calcium salt may be administered at 0.05 to 20 mg/day, preferably at 0.5 to 10 mg/day, and more preferably at 1 to 4 mg/day; in one dose, two doses, or if desired in several doses per day. Depending on the physician's indication, a daily dose lower than the physician's recommendation (for example, 1 to 10 mg in the case of lovastatin) may be orally administered on the first day of the administration, and thereafter, a dose gradually increasing to the daily maximum dose (of up to 80 mg in the case of lovastatin) may be orally administered as the maintenance dose. The dose of the second ingredient may also be reduced depending on the dose of the first ingredient. In view of reducing the side effect, preferred is the use of minimized daily dose and administration of once a day. When such dose is orally administered, the administration period is at least 1 year, preferably at least 2 years, more preferably at least 3.5 years, still more preferably at least 5 years. However, the administration is continued as long as pathological conditions or biochemical index related to NASH or high risk of onset and/or recurrence of the NASH is recognized. Possible embodiments also include administration on every alternate day, administration of 2 to 3 days per week, and inclusion of a washout period of about 1 day to 3 months, and preferably 1 week to 1 month.

When the second ingredient is orally administered in the fourth aspect of the present invention, for example, losartan potassium salt may be administered at 1 to 500 mg/day, preferably at 10 to 250 mg/day, and more preferably at 25 to 100 mg/day; valsartan may be administered at 2 to 1600 mg/day, preferably at 20 to 800 mg/day, and more preferably at 40 to 320 mg/day; irbesartan may be administered at 2 to 1500 mg/day, preferably at 20 to 750 mg/day, and more preferably at 50 to 300 mg/day; eprosartan mesylate may be administered at 20 to 4000 mg/day, preferably at 200 to 2000 mg/day, and more preferably at 400 to 800 mg/day; candesartan cilexetil may be administered at 0.2 to 160 mg/day, preferably at 2 to 80 mg/clay, and more preferably at 4 to 32 mg/day; telmisartan may be administered at 1 to 400 mg/day, preferably at 10 to 200 mg/day, and more preferably at 20 to 80 mg/day; and olmesartan medoxomil may be administered at 0.5 to 200 mg/day, preferably 5 to 100 mg/day, and more preferably 10 to 40 mg/day; in one dose, two doses, or if desired, in several doses per day. Depending on the physician's indication, a daily dose lower than the physician's recommendation (for example, 1 to 20 mg in the case of losartan potassium salt) may be orally administered on the first day of the administration, and thereafter, a dose gradually increasing to the daily maximum dose (of up to 150 mg in the case of losartan potassium salt) may be orally administered as the maintenance dose. The dose of the second ingredient may also be reduced depending on the dose of the first ingredient. In view of reducing the side effect, preferred is the use of minimized daily dose. When such dose is orally administered, the administration period is at least 1 year, preferably at least 2 years, more preferably at least 3.5 years, still more preferably at least 5 years. However, the administration is continued as long as pathological conditions or biochemical index related to NASH or high risk of onset and/or recurrence of the NASH is recognized. Possible embodiments also include administration on every alternate day, administration of 2 to 3 days per week, and inclusion of a washout period of about 1 day to 3 months, and preferably 1 week to 1 month.

In the present invention, the first ingredient and the second ingredient are used in combination, and this enables use of the first ingredient and/or the second ingredient at a dose lower than the conventional dose which is generally used for the particular drug. For example, the dose of the first ingredient and/or the second ingredient may be the one which would be insufficient for realizing the intended therapeutic effect if each ingredient was used alone. Use of such dose has the merit of reducing the side effects of the drugs, and in particular, the side effects induced by the second ingredient.

A preferable embodiment of the use of the present invention is an embodiment such that the dose of the first ingredient and/or the second ingredient is a dose which would be insufficient for realizing the intended therapeutic effects when each ingredient was used alone, but such that the therapeutic effect realized by the combination of the first ingredient and the second ingredient is higher than the sum of the therapeutic effect that would have been achieved when the first and second ingredients were separately used at the same dose.

Another preferable embodiment of the use of the present invention is an embodiment such that the dose of the first ingredient and/or the second ingredient is a dose which would be insufficient for realizing the intended therapeutic effects when each ingredient was used alone, but such that the side effect induced by the combination of the first ingredient and the second ingredient is lower than the sum of the side effect that would have been achieved when the first and second ingredients were separately used at the same dose.

The dose of the first ingredient which is insufficient for establishing the therapeutic effects when this ingredient was used alone is not particularly limited since such dose depends on the conditions and body type of the individual patient. However, exemplary such dose (daily close) in the case of EPA-E and/or DHA-E is at least 0.1 g and less than 2 g, preferably at least 0.2 g and up to 1.8 g, more preferably at least 0.3 g and less than 0.9 g, and most preferably at least 0.3 g and up to 0.6 g.

In the first aspect of the present invention, the dose of the second ingredient, namely, biguanide hypoglycemic agent which is insufficient for establishing the therapeutic effects when this ingredient was used alone is not particularly limited since such dose depends on the conditions and body type of the individual patient. However, exemplary such dose (daily dose) in the case of metformin hydrochloride is less than the recommended daily dose of 500 mg/day, preferably at least 10 mg and up to 400 mg, more preferably at least 20 mg and up to 300 mg, and still more preferably at least 50 mg and up to 200 mg; and in the case of buformin hydrochloride, less than the recommended daily dose of 100 mg/day, preferably at least 2 mg and up to 80 mg, more preferably at least 4 mg and up to 60 mg, and still more preferably at least 10 mg and up to 40 mg.

The ratio of the dose of the first ingredient to the dose of the biguanide hypoglycemic agent is not particularly limited. The ratio of the dose of the first ingredient to the dose of the biguanide hypoglycemic agent when the first ingredient is metformin hydrochloride is 1 to 50:1, preferably 1.5 to 20:1, more preferably 2 to 10:1, and most preferably 2.5 to 3:1; and the ratio when the first ingredient is buformin hydrochloride is 5 to 250:1, preferably B to 100:1, more preferably 10 to 50:1, and most preferably 12 to 15:1. In view of reducing the side effects by the biguanide hypoglycemic agent, the dose of the biguanide hypoglycemic agent may be further reduced to ½ to ⅕. In the case of human, metformin hydrochloride may be used at 250 to 750 mg, and preferably 500 mg, and buformin hydrochloride may be used at 50 to 150 mg, and preferably 100 mg in relation to 1800 mg of the first ingredient. In view of reducing the side effects, the metformin hydrochloride may be used at 100 to 250 mg and the buformin hydrochloride may be used at 20 to 50 mg in relation to 1800 mg of the first ingredient. When the ingredients are formulated in the form of a composite formulation, the ingredients are preferably combined at such ratio.

Daily dose, number of administration per day, or dosage rate of the biguanide hypoglycemic agent and the first ingredient may be adequately adjusted by confirming test results such as degree of liver fibrosis, decrease in the serum AST or ALT, decrease in the AST/ALT ratio, increase in the adiponectin, decrease in the TNFα, decrease in the blood oxidative stress marker, and improvement in the HOMA-IR. For example, serum ALT level may be measured for the sole administration of the biguanide hypoglycemic agent, and the result of this measurement may be used as the index in the subsequent administration in which the administration of the first ingredient is started by reducing the dose of the biguanide hypoglycemic agent to thereby realize the therapeutic effect of the present invention. It is preferable that the side effects, for example, increase in the plasma lactic acid level developed after the administration of the prophylactic/ameliorating or therapeutic agent of the present invention do not exceed the side effects when the biguanide hypoglycemic agent is administered alone at the dose required for realizing the equivalent therapeutic effects.

In the second aspect of the present invention, the dose of the second ingredient, namely, acetylsalicylic acid which is insufficient for establishing the therapeutic effects when this ingredient is used alone is not particularly limited since such dose depends on the conditions and body type of the individual patient. However, exemplary such dose (daily dose) is less than the recommended daily dose of 500 mg/day, preferably at least 10 mg and up to 300 mg, more preferably at least 20 mg and up to 200 mg, and still more preferably at least 50 mg and up to 100 mg.

In the present invention, the desired effect is expected to be realized at a dose lower than the dose required for the acetylsalicylic acid to develop the anti-inflammatory effect or analgesic effect when acetylsalicylic acid is used alone. For example, the effect is expected to be realized at a lower dose of the level required for realizing the anti-platelet action.

The ratio of the dose of the first ingredient to the dose of the acetylsalicylic acid is not particularly limited. The ratio of the dose of the first ingredient to the close of the acetylsalicylic acid when the first ingredient is acetylsalicylic acid may be 0.5 to 100:1, preferably 1 to 50:1, more preferably 1.5 to 20:1, and most preferably 2 to 10:1; and in view of reducing the side effects by the acetylsalicylic acid, the dose of the acetylsalicylic acid may be further reduced to ½ to 1/10. In the case of human, acetylsalicylic acid may be used at 500 to 1500 mg, and preferably at 1000 mg in relation to 1800 mg of the first ingredient. In view of reducing the side effect, the acetylsalicylic acid may be used at 50 to 500 mg in relation to 1800 mg of the first ingredient. When the ingredients are formulated in the form of a composite formulation, the ingredients are preferably combined at such ratio.

Daily dose, number of administration per day, or dosage rate of the acetylsalicylic acid and the first ingredient may be adequately adjusted by confirming test results such as decrease in the serum AST or ALT level, decrease in the AST/ALT ratio, increase in the adiponectin, decrease in the TNFα, IL, or high sensitivity CPR, blood oxidative stress marker, neutrophil count. For example, serum ALT level may be measured for the sole administration of the acetylsalicylic acid, and the result of this measurement may be used as the index in the subsequent administration in which the administration of the first ingredient is started by reducing the dose of the acetylsalicylic acid to thereby realize the therapeutic effect of the present invention. It is preferable that the side effects, for example increase in the plasma AST or ALT level, developed after the administration of the prophylactic/ameliorating or therapeutic agent of the present invention do not exceed the side effects when the acetylsalicylic acid is administered alone at the dose required for realizing the equivalent therapeutic effects.

In the third aspect of the present invention, the dose of the second ingredient, namely, HMG-CoA reductase inhibitor which is insufficient for establishing the therapeutic effects when this ingredient was used alone is not particularly limited since such dose depends on the conditions and body type of the individual patient. However, exemplary such dose (daily dose) in the case of lovastatin is less than the recommended daily dose of 10 mg/day, preferably at least 0.2 mg and up to 8 mg, more preferably at least 0.4 mg and up to 6 mg, and still more preferably at least 1 mg and up to 4 mg; in the case of pravastatin sodium salt, less than the recommended daily dose of 10 mg/day, preferably at least 0.2 mg and up to 8 mg, more preferably at least 0.4 mg and up to 6 mg, and still more preferably at least 1 mg and up to 4 mg; in the case of simvastatin, less than the recommended daily dose of 1 day 5 mg, preferably at least 0.1 mg and up to 4 mg, more preferably at least 0.2 mg and up to 2 mg, and still more preferably at least 0.4 mg and up to 1 mg; in the case of fluvastatin sodium salt, less than the recommended daily dose of 20 mg/day, preferably at least 0.4 mg and up to 16 mg, more preferably at least 0.8 mg and up to 12 mg, and still more preferably at least 1.5 mg and up to 8 mg; in the case of atorvastatin calcium hydrate, less than the recommended daily dose of 10 mg/day, preferably at least 0.2 mg and up to 8 mg, more preferably at least 0.4 mg and up to 6 mg, and still more preferably at least 1 mg and up to 4 mg; in the case of rosuvastatin calcium salt, less than the recommended daily dose of 2.5 mg, preferably at least 0.05 mg and up to 2 mg, more preferably at least 0.1 mg and up to 1.5 mg, and still more preferably at least 0.2 mg and up to 1 mg; and in the case of pitavastatin calcium salt, less than the recommended daily dose of 1 day 1 mg, preferably at least 0.02 mg and up to 0.8 mg, more preferably at least 0.04 mg and up to 0.6 mg, and still more preferably at least 0.1 mg and up to 0.4 mg.

In the present invention, the desired effect is expected to be realized at a dose lower than the dose required for the HMG-CoA reductase inhibitor to have the serum lipid lowering action when HMG-CoA reductase inhibitor is used alone.

The ratio of the dose of the first ingredient to the dose of the HMG-CoA reductase inhibitor is not particularly limited. The ratio of the dose of the first ingredient to the dose of the HMG-CoA reductase inhibitor when the first ingredient is lovastatin is 5 to 2000:1, preferably 10 to 1000:1, more preferably 15 to 400:1, and most preferably 25 to 150:1; the ratio when the first ingredient is pravastatin sodium salt is 5 to 2000:1, preferably 10 to 1000:1, more preferably 15 to 400:1, and most preferably 25 to 150:1; the ratio when the first ingredient is simvastatin is 5 to 5000:1, preferably 10 to 3000:1, more preferably 15 to 1000:1, and most preferably 25 to 400:1; the ratio when the first ingredient is fluvastatin sodium salt is 5 to 1000:1, preferably 10 to 600:1, more preferably 15 to 200:1, and most preferably 25 to 80:1; the ratio when the first ingredient is atorvastatin calcium hydrate is 5 to 2000:1, preferably 10 to 1000:1, more preferably 15 to 400:1, and most preferably 25 to 150:1; the ratio when the first ingredient is rosuvastatin calcium salt is 10 to 10000:1, preferably 20 to 5000:1, more preferably 30 to 1000:1, and most preferably 40 to 500:1; and the ratio when the first ingredient is pitavastatin calcium salt is 50 to 10000:1, preferably 100 to 5000:1, more preferably 200 to 3000:1, and most preferably 500 to 1200:1. In view of reducing the side effects by the HMG-CoA reductase inhibitor, the dose of the HMG-CoA reductase inhibitor may be further reduced to ½ to ⅕. In the case of human, the lovastatin may be used at 25 to 50 mg, the pravastatin sodium salt may be used at 40 to 80 mg, the simvastatin may be used at 50 to 100 mg, the fluvastatin sodium salt may be used at 400 to 800 mg, the atorvastatin calcium hydrate may be used at 4 to 8 mg, the rosuvastatin calcium salt may be used at 20 to 40 mg, and the pitavastatin calcium salt may be used at 10 to 20 mg in relation to 1800 mg of the first ingredient. When the ingredients are formulated in the form of a composite formulation, the ingredients are preferably combined at such ratio.

Daily dose, number of administration per day, or dosage rate of the HMG-CoA reductase inhibitor and the first ingredient may be adequately adjusted by confirming test results such as decrease in the serum AST and ALT level, decrease in the AST/ALT ratio, decrease in the TNFα, IL, or high sensitivity CPR, blood oxidative stress marker (such as ferritin or thioredoxin), fibrosis marker (such as type IV collagen, hyaluronic acid, or TIMP-1), or serum CPK. For example, serum ALT level may be measured for the sole administration of the HMG-CoA reductase inhibitor, and the result of this measurement may be used as the index in the subsequent administration in which the administration of the first ingredient is started by reducing the dose of the HMG-CoA reductase inhibitor to thereby realize the therapeutic effect of the present invention. It is preferable that the side effects, for example increase in the serum CPK, developed after the administration of the prophylactic/ameliorating or therapeutic agent of the present invention do not exceed the side effects when the HMG-CoA reductase inhibitor is administered alone at the dose required for realizing the equivalent therapeutic effects.

In the fourth aspect of the present invention, the dose of the second ingredient, namely, ARB which is insufficient for establishing the therapeutic effects when this ingredient is used alone is not particularly limited since such dose depends on the conditions and body type of the individual patient. However, exemplary such dose (daily dose) in the case of losartan potassium salt is less than the recommended daily dose of 25 mg/day, preferably at least 0.5 mg and up to 20 mg, more preferably at least 1 mg and up to 15 mg, and still more preferably at least 2 mg and up to 10 mg; in the case of valsartan, less than the recommended daily dose of 40 mg/day, preferably at least 1 mg and up to 30 mg, more preferably at least 2 mg and up to 20 mg, and still more preferably at least 4 mg and up to 10 mg; in the case of irbesartan, less than the recommended daily dose of 50 mg/day, preferably at least 1 mg and up to 40 mg, more preferably at least 2 mg and up to 30 mg, and still more preferably at least 5 mg and up to 20 mg; in the case of eprosartan mesylate, less than the recommended daily dose of 400 mg/day, preferably at least 10 mg and up to 300 mg, more preferably at least 20 mg and up to 200 mg, and still more preferably at least 30 mg and up to 100 mg; in the case of candesartan cilexetil, less than the recommended daily dose of 4 mg/day, preferably at least 0.1 mg and up to 3 mg, more preferably at least 0.2 mg and up to 2 mg, and still more preferably at least 0.4 mg and up to 1 mg; in the case of telmisartan, less than the recommended daily dose of 20 mg/day, preferably at least 0.5 mg and up to 15 mg, more preferably at least 1 mg and up to 10 mg, and still more preferably at least 2 mg and up to 5 mg; and in the case of olmesartan medoxomil, less than the recommended daily dose of 10 mg/day, preferably at least 0.2 mg and up to 8 mg, more preferably at least 0.5 mg and up to 6 mg, and still more preferably at least 1 mg and up to 4 mg.

In the present invention, the desired effect is expected to be realized at a dose lower than the dose required for the ARB to have the hypotensive action when ARB is used alone.

The ratio of the dose of the first ingredient to the dose of the ARB is not particularly limited. The ratio of the dose of the first ingredient to the dose of the ARB when the first ingredient is losartan potassium salt is 10 to 1000:1, preferably 20 to 500:1, more preferably 30 to 200:1, and most preferably 40 to 60:1; the ratio when the first ingredient is valsartan is 5 to 500:1, preferably 10 to 300:1, more preferably 15 to 100:1, and most preferably 25 to 40; the ratio when the first ingredient is irbesartan is 5 to 500:1, preferably 10 to 300:1, more preferably 15 to 100:1, and most preferably 20 to 30:1; the ratio when the first ingredient is eprosartan mesylate is 0.5 to 50:1, preferably 1 to 30:1, more preferably 2 to 10:1, and most preferably 2.5 to 4:1; the ratio when the first ingredient is candesartan cilexetil is 0.5 to 50:1, preferably 1 to 30:1, more preferably 2 to 10:1, and most preferably 4 to 6:1; the ratio when the first ingredient is telmisartan is 10 to 1000:1, preferably 20 to 600:1, more preferably 30 to 200:1, and most preferably 50 to 80:1; and the ratio when the first ingredient is olmesartan medoxomil is 20 to 2000:1, preferably 40 to 1200:1, more preferably 60 to 400:1, and most preferably 100 to 150:1. In view of reducing the side effect of ARB, the dose of the ARB may be further reduced to ½ to ⅕. In the case of human, the losartan potassium salt may be used at 25 to 50 mg, and preferably at 25 mg; the valsartan may be used at 40 to 80 mg, and preferably at 40 mg; the irbesartan may be used at 50 to 100 mg, and preferably at 50 mg; the eprosartan mesylate may be used at 400 to 800 mg, and preferably at 400 mg; the candesartan cilexetil may be used at 4 to 8 mg, and preferably at 4 mg, the telmisartan may be used at 20 to 40 mg, and preferably at 20 mg, and the olmesartan medoxomil may be used at 10 to 20 mg, and preferably at 10 mg in relation to 1800 mg of the first ingredient. In view of reducing the side effect, the losartan potassium salt may be used at 5 to 25 mg, the valsartan may be used at 8 to 40 mg, the irbesartan may be used at 20 to 50 mg, the eprosartan mesylate may be used at 160 to 400 mg, the candesartan cilexetil may be used at 1.6 to 4 mg, may be used at telmisartan 8 to 20 mg, and the olmesartan medoxomil may be used at 4 to 10 mg in relation to 1800 mg of the first ingredient. When the ingredients are formulated in the form of a composite formulation, the ingredients are preferably combined at such ratio.

Daily dose, number of administration per day, or dosage rate of the ARB and the first ingredient may be adequately adjusted by confirming test results such as degree of liver fibrosis, decrease in the serum AST or ALT, decrease in the AST/ALT ratio, increase in the adiponectin, decrease in the TNFα, decrease in the blood oxidative stress marker, improvement in the HOMA-IR. For example, serum ALT level may be measured for the sole administration of the ARB, and the result of this measurement may be used as the index in the subsequent administration in which the administration of the first ingredient is started by reducing the dose of the ARB to thereby realize the therapeutic effect of the present invention. It is preferable that the side effects, for example, decrease in the transient hypotension developed after the administration of the prophylactic/ameliorating or therapeutic agent of the present invention do not exceed the side effects when the ARB is administered alone at the dose required for realizing the equivalent therapeutic effects.

In administering the prophylactic/ameliorating or therapeutic agent for NASH of the present invention, the active ingredients may be administered either as-prepared compounds (which may contain inevitable ingredients remaining after the purification), or after preparing into an adequate medical preparation by suitably combining with an adequate commonly used carrier, medium, excipient, binder, lubricant, colorant, flavor, or optional additives such as sterilized water, vegetable oil, non-toxic organic solvent, non-toxic solubilizing agent (for example, glycerin or propylene glycol), emulsifier, suspending agent (for example, Tween 80 or gum arabic solution), isotonic agent, pH adjusting agent, stabilizer, soothing agent, corrective, flavoring agent, preservative, antioxidant, buffer, or colorant. Specific examples of the additive include lactose, partially gelatinization starch, hydroxypropyl cellulose, macrogol, tocopherol, hydrogenated oil, sucrose fatty ester, hydroxypropyl methylcellulose, titanium oxide, talc, dimethylpolysiloxane, silicon dioxide, and carnauba wax.

Since the first ingredient is highly unsaturated, incorporation of an effective amount of an antioxidant, for example, at least one member selected from butylated hydroxytoluene, butylated hydroxyanisole, propyl gallate, gallic acid, pharmaceutically acceptable quinine, and α-tocopherol is particularly preferable.

In addition, in the second aspect of the present invention, an antacid such as dialuminate may be added to the drug or the drug may be formed with an enteric coating by providing an acid-resistant film to thereby reduce the side effects of the acetylsalicylic acid on the digestive tract.

The dosage form of the preparation is not particularly limited since the dosage form may differ by the way how the active ingredients of the present invention are combined. Exemplary dosage forms in the case of oral preparation include tablet, film coated tablet, capsule, microcapsule, granules, fine granules, powder, oral liquid preparation, syrup, jelly, and inhalant, and exemplary dosage forms for parenteral preparation include ointment, suppository, injection (emulsion, suspension, or non-aqueous), or solid injection which is emulsified or suspended before use, infusion, and external medicine, for example, for percutaneous absorption. While the drug may be administered orally, intravenously, intraarterially, by inhalation, rectally, intravaginally, or by external administration, when oral administration is possible, oral dosage form is desirable in view of the administration convenience, and the preferred is the oral administration by capsule such as soft capsule or microcapsule having the drug incorporated therein, and the administration by way of tablet or film coated tablet. Oral administration by using an enteric-coated preparation or extended release preparation is also preferable, and use of jelly for oral administration is also preferable for patients undergoing dialysis or patients suffering from aphagia.

The prophylactic/ameliorating or therapeutic agent of the present invention may also be realized by combined use of two separately formulated preparations, and in such case, they may be formulated by a method known in the art. The prophylactic/ameliorating or therapeutic agent of the present invention may also be formulated as a composite formulation containing wherein the first ingredient and the second ingredient are the active ingredients.

The composite formulation may also have a third drug incorporated therein as the active ingredient, and while the third drug is not particularly limited to any particular type, the third drug is preferably the one which does not reduce the merits of the present invention. Exemplary such agents include liver protectant, hypoglycemic drug, antihyperlipidemia drug, antihypertensive agent, antioxidant, and anti-inflammatory agent, and any suitable drug which is not the second ingredient in the first to fourth aspect of the invention may be used for the third drug.

Exemplary liver protectants include ursodeoxycholic acid and betaine. Exemplary hypoglycemic drug include insulin and insulin derivatives, sulfonylurea drugs such as tolbutamide, gliclazide, glibenclamide, and glimepiride, prompt insulin secretagogues such as nateglinide, repaglinide, and mitiglinide, α glucosidase inhibitors such as acarbose, voglibose, and miglitol, thiazolidines such as pioglitazone, rosiglitazone, and troglitazone, and biguanide hypoglycemic agent such as metformin and buformin. Exemplary antihyperlipidemia agents include HMG-CoA reductase inhibitors such as pravastatin, simvastatin, atorvastatin, fluvastatin, pitavastatin, rosuvastatin, and cerivastatin; fibrate drugs such as simfibrate, clofibrate, clinofibrate, bezafibrate, and fenofibrate; lipase inhibitors such as orlistat; and ezetimibe. Exemplary antihypertensive agents include angiotensin converting enzyme inhibitors such as captopril, alacepril, imidapril, enalapril, cilazapril, temocapril, delapril, lisinopril, and benazepril; angiotensin receptor antagonists such as losartan, valsartan, candesartan, telmisartan, olmesartan, irbesartan, and eprosartan; renin inhibitors such as aliskiren; calcium antagonists such as amlodipine, nifedipine, benidipine, nicardipine, nilvadipine, cilnidipine, azelnidipine, manidipine, nitrendipine, barnidipine, nisoldipine, efonidipine, felodipine, aranidipine, diltiazem, verapamil, and bepridil. Exemplary antioxidants include vitamins such as vitamin C and vitamin E; N-acetylcysteine, and probucol. Exemplary anti-inflammatory agents include cytokine production inhibitors such as pentoxifylline; leukotriene receptor antagonist, leukotriene synthesis inhibitor, NSAIDs, COX-2-selective inhibitor, M2/M3 antagonist, steroids such as corticosteroid and prednisolone farnesylate; Hi(histamine) receptor antagonist, salazosulfapyridine; and aminosalicylic acids such as mesalazine. Exemplary immunosuppressants include azathioprine, 6-mercaptopurine, and tacrolimus. Exemplary antiviral agents for hepatitis C virus (HCV) include interferon, protease inhibitor, helicase inhibitor, and polymerase inhibitor.

The composite formulation is not particularly limited for its dosage form, and it may be administered in the form of an oral preparation such as tablet, film coated tablet, capsule, microcapsule, granules, fine granules, powder, oral liquid preparation, syrup, or jelly, or in the form of parenteral preparations such as injection, infusion, percutaneous absorptive preparation, or other external medicine. The composite formulation may also be an extended release preparation, or a preparation in which the active ingredients are released at different timing.

The composite formulation of the present invention may contain a pharmaceutically acceptable excipient in addition to the active ingredients, and any known antioxidant, coating agent, gelation agent, corrective, flavoring agent, preservative, antioxidant, emulsifier, pH adjusting agent, buffer, colorant, or the like may be incorporated as required.

The composite formulation of the present invention may be prepared by a method commonly used in the art. More specifically, the powder of the first ingredient may be prepared by a method commonly used in the art, for example, by drying an oil-in-water emulsion containing (A) EPA-E, (B) a dietary fiber, (C) a starch hydrolysate and/or a low saccharification reduced starch decomposition product, and (D) water soluble antioxidant under high vacuum, and pulverizing the dried emulsion (JP A 10-99046). Granules, fine granules, powder, tablet, film coated tablet, chewable tablet, extended release tablet, or orally disintegrating tablet (OD tablet) can be produced using the thus obtained EPA-E powder and the second ingredient by the method commonly used in the art. In the case of the chewable tablet, the tablet may be prepared by the method known in the art, for example, by emulsifying EPA-E in a water-soluble polymer such as hydroxypropylmethylcellulose, and spraying the resulting emulsion onto an additive such as lactose to obtain a granule powder (JP A 8-157362), mixing this granule powder with the second ingredient, and producing the tablet. In the case of the extended release tablet, the tablet may be prepared, for example, (1) by forming an inner core of either one of the EPA-E and the second ingredient and coating the inner core with an outer layer which is either one of the EPA-E and the second ingredient which was not used for the inner core; (2) by placing a disk-shaped matrix containing one ingredient on another disk-shaped matrix containing the other ingredient; (3) by embedding a granular capsule containing one ingredient in a matrix containing the other ingredient; or (4) by preliminarily mixing both ingredients, and thereafter providing some kind of extended release mechanism. The active ingredients are preferably regulated for their release speed, and both ingredients may be released simultaneously or in sequential manner with time lag. The drug may be prepared by a method known in the art, and an orally disintegrating tablet may be prepared by a method disclosed, for example, in JP A 8-333243, and a film preparation for oral administration may be prepared by a method disclosed, for example, in JP A 2005-21124. Since the second ingredient is not easily soluble in the first ingredient, ideas described in the Examples would be needed in the production of a soft capsule or liquid preparation. The composite formulation of the present invention also includes drugs prepared by using such ideas for incorporating the first ingredient and the second ingredient in the same drug.

The composite formulation of the present invention is preferably released and absorbed so that the pharmacological actions of the active ingredients are developed. The composite formulation of the present invention may preferably have at least one merit selected from improved releasability of the active ingredient, improved absorption properties of the active ingredient, improved dispersibility of the active ingredient, improved storage stability of the composite formulation; ease of taking the drug, and improved compliance of the preparation.

The prophylactic/ameliorating or therapeutic agent of the present invention is effective for prevention/amelioration, treatment, secondary prevention, and prevention of the progress into the liver cirrhosis of NAFLD, and in particular, NASH of an animal, and in particular, a mammal. Exemplary mammals include human, domestic animals such as cattle, horse, and pig, and companion animals such as dog, cat, rabbit, rat, and mouse, and the preferred is human.

The prophylactic/ameliorating or therapeutic agent of the present invention is expected to exhibit synergistic prophylactic/ameliorative or therapeutic effects in NASH patients who have developed insulin resistance, NASH patients with reduced adiponectin, or NASH patients with increased TNFα including the patients suffering from diabetes or metabolic syndrome of the first aspect of the present invention.

The prophylactic/ameliorating or therapeutic agent of the present invention is capable of reducing side effects in NASH patient who are expected to suffer from lactic acidosis of delayed hypoglycemia by the administration of biguanide hypoglycemic agent and the like. The prophylactic/ameliorating or therapeutic agent of the present invention can also be administered to the patients who could not receive the treatment or the patients who had to stop the treatment because of the side effects of the biguanide hypoglycemic agent.

The present invention is capable of ameliorating the side effects in the NASH patient in the second aspect of the present invention who have the risk of side effects such as aspirin hypersensitivity and gastrointestinal disorder induced by acetylsalicylic acid, and therefore, the present invention enables treatment with the acetylsalicylic acid of the patients who could not receive the treatment or the patients who had to stop the treatment because of such side effects. The present invention is particularly useful in patients suffering from angina pectoris, myocardial infarction, or ischemic or cerebrovascular disease, and patients who have experienced coronary artery bypass grafting or percutaneous transluminal coronary angioplasty who are in need of continued administration of the acetylsalicylic acid.

The prophylactic/ameliorating or therapeutic agent of the present invention is expected to exhibit synergistic prophylactic/ameliorative or therapeutic effects in NASH patients who have increased blood lipid, NASH patients who have developed insulin resistance, NASH patients with increased TNFα, or NASH patients with increased fibrosis marker including the patients suffering from hyperlipemia or metabolic syndrome in the third aspect of the present invention. The agent of the present invention is also capable of reducing the side effects in the NASH patients who have the risk of rhabdomyolysis or increase of the serum CPK by the HMG-CoA reductase inhibitor, and therefore, the present invention enables treatment of the patients who could not receive the treatment or the patients who had to stop the treatment with the HMG-CoA reductase inhibitor because of such side effects.

The prophylactic/ameliorating or therapeutic agent of the present invention is expected to exhibit synergistic prophylactic/ameliorative or therapeutic effects in NASH patients who have developed insulin resistance and increased fibrosis marker (such as or example, type IV collagen, hyaluronic acid, and TIMP-1) in the fourth aspect of the present invention. The agent of the present invention is also capable of reducing the side effects by the ARB in the NASH patients suffering from renal or liver dysfunction, for example, the patient suffering from bilateral renal artery stenosis, and therefore, the present invention enables treatment of the patients who could not receive the treatment or the patients who had to stop the treatment with the ARB because of such side effects.

Furthermore, the present invention is capable of further improving the prophylactic/ameliorative or therapeutic effects by reducing the burden of the patients by providing the agent in the form of a composite formulation or a kit and thereby improving the drug compliance.

### EXAMPLES

Next, the present invention is described in further detail, which by no means limit the scope of the present invention.

### Experimental Example 1

### Effectiveness in Rats Fed with Methionine-Choline-Deficient Diet

Pharmacological action of EPA-E and/or metformin hydrochloride, acetylsalicylic acid, simvastatin, or valsartan on liver disorder and fibrosis is confirmed by using rats that have been fed with methionine-choline-deficient diet (hereinafter referred to as “MCD diet”) known to develop a NASH-like lesion in the liver.

7 week old male Wister rats are allowed to freely take normal diet (F-1, Funabashi Farm) or MCD diet (Dyets, Inc.) for 20 weeks under 12 hour light-dark cycles at 23° C. 11 groups (each comprising 20 animals), namely, normal group (normal diet feeding); control group (MCD diet feeding); EPA-E group (MCD diet feeding+EPA-E administration); metformin group (MCD diet feeding+metformin hydrochloride administration) and metformin combination group (MCD diet feeding+EPA-E administration+metformin hydrochloride administration); acetylsalicylic acid group (MCD diet feeding+acetylsalicylic acid administration) and acetylsalicylic acid combination group (MCD diet feeding+EPA-E administration+acetylsalicylic acid administration); simvastatin group (MCD diet feeding+simvastatin administration) and simvastatin combination group (MCD diet feeding+EPA-E administration+simvastatin administration); valsartan group (MCD diet feeding+valsartan administration), and valsartan combination group (MCD diet feeding+EPA-E administration+valsartan administration) are set. During the feeding, the EPA-E group is administered with 1000 mg/kg of EPA-E; the metformin group is administered with 40 mg/kg of metformin hydrochloride; the metformin combination group is administered with 1000 mg/kg of EPA-E and 40 mg/kg of metformin hydrochloride; the acetylsalicylic acid group is administered with 20 mg/kg of acetylsalicylic acid; the acetylsalicylic acid combination group is administered with 1000 mg/kg of EPA-E and 20 mg/kg of acetylsalicylic acid; the simvastatin group is administered with 5 mg/kg of simvastatin; the simvastatin combination group is administered with 1000 mg/kg of EPA-E and 5 mg/kg of simvastatin; the valsartan group is administered with 20 mg/kg of valsartan; and the valsartan combination group is administered with 1000 mg/kg of EPA-E and 20 mg/kg of valsartan after suspending in 5% aqueous solution of gum arabic. The administration is conducted orally once a day. The normal group and the control group are orally administered with 5% aqueous solution of gum arabic once a day. After 20 weeks, blood is collected for biochemical tests of the plasma, and pathological tests of the liver are also conducted.

Compared to the normal group, the control group has significantly increased plasma AST, ALT, total bilirubin, albumin, total protein, choline esterase, type IV collagen, hyaluronic acid, and TIMP-1 content as well as significantly increased fibrosis area in the liver by Masson's trichrome stain and significantly increased hydroxyproline content with NASH-like liver lesion.

Compared to the control group, the EPA-E group exhibits suppressed increase of the plasma AST, ALT, total bilirubin, albumin, total protein, choline esterase, type Iv collagen, hyaluronic acid, and TIMP-1 content as well as increase of fibrosis area of liver and hydroxyproline content.

Compared to the control group, the metformin group, the simvastatin group, and the valsartan group exhibits effects such as suppression of the increase of the plasma ALT similar to the EPA-E group. In contrast, the acetylsalicylic acid does not exhibit effects like those of the EPA-E group, and some parameters such as plasma ALT and AST are exacerbated. The effect of suppressing the increase of plasma biochemical parameters found in the metformin combination group, the simvastatin combination group, and the valsartan combination group is larger than the sum of the effects realized in each of the EPA-E group, the metformin group, the simvastatin group, and the valsartan group. In addition, the effects of suppressing the increase of the parameters found in the acetylsalicylic acid combination group is larger than the effect found in the EPA-E group, and the exacerbation effect found in the acetylsalicylic acid group is suppressed in the acetylsalicylic acid combination group. Accordingly, the prophylactic/ameliorating or therapeutic agent of the present invention is useful for prevention/amelioration or treatment of the NASH.

### Experimental Example 2

### Effectiveness in Methionine-Choline Deficient Diet Diabetes Model Mouse

Pharmacological action of EPA-E and/or metformin hydrochloride or olmesartan medoxomil for liver disorder and fibrosis is confirmed by using diabetes model rats that have been fed with MCD diet known to induce a NASH-like lesion in the liver.

7 week old male db/db mouse (Charles River Japan) is allowed to freely take normal diet (F-1, Funabashi Farm) or MCD diet (Dyets, Inc.) for 2 weeks under 12 hour light-dark cycles at 23° C. 7 groups (each comprising 20 animals), namely, normal group (normal diet feeding), control group (MCD diet feeding), EPA-E group (MCD diet feeding+EPA-E administration), metformin group (MCD diet feeding+metformin hydrochloride administration), metformin combination group (MCD diet feeding+EPA-E administration+metformin hydrochloride administration), olmesartan group (MCD diet feeding+olmesartan medoxomil administration), and olmesartan combination group (MCD diet feeding+EPA-E administration olmesartan medoxomil administration) are set. During the feeding, the EPA-E group is administered with 1000 mg/kg of EPA-E; the metformin group is administered with 80 mg/kg of metformin hydrochloride; the metformin combination group is administered with 1000 mg/kg of EPA-E and 80 mg/kg of metformin hydrochloride; the olmesartan group is administered with 5 mg/kg of olmesartan medoxomil; and the olmesartan combination group is administered with 1000 mg/kg of EPA-E and 5 mg/kg of olmesartan medoxomil, after suspending in 5% aqueous solution of gum arabic. The administration is conducted orally once a day. The normal group and the control group are orally administered with 5% aqueous solution of gum arabic once a day. After 2 weeks, HOMA-IR is measured and blood is collected for biochemical test of the plasma.

Compared to the normal group, the control group exhibits exacerbated HOMA-IR, significantly increased plasma AST, ALT, type IV collagen, hyaluronic acid, TIMP-1 and FFA amount, reduced plasma adiponectin, and increased TNFα.

Compared to the control group, EPA-E group exhibits suppressed increase of the plasma AST, ALT, type IV collagen, hyaluronic acid, TIMP-1, and FFA amount, suppressed decrease of the adiponectin amount, and suppressed increase of the TNFα.

Compared to the control group, the metformin group exhibits improved HOMA-IR and suppressed increase of the plasma ALT and FFA amount; and the olmesartan group exhibits similar effects as the EPA-E group. With regard to the effects of improving the parameters, the metformin combination group and the olmesartan combination group exhibit effects which are larger than the sum of the effects realized in each of the EPA-E group, the metformin group, and olmesartan group. Accordingly, the prophylactic/ameliorating or therapeutic agent of the present invention is useful for prevention/amelioration or treatment of the NASH.

### Experimental Example 3

### Effectiveness in High Fat Diet-Fed Rat

4 week old male SD rat is allowed to freely take normal diet (F-1, Funabashi Farm) or high-fat high-sucrose diet (TD88137, Harlan Teklad, hereinafter referred to as “HF diet”) for 4 weeks under 12 hour light-dark cycles at 23° C. 7 groups (each comprising 10 animals), namely, normal group (normal diet feeding), control group (HF diet feeding), EPA-E group (HF diet feeding+EPA-E administration), acetylsalicylic acid group (HF diet feeding+acetylsalicylic acid administration), acetylsalicylic acid combination group (HF diet feeding+EPA-E administration+acetylsalicylic acid administration), atorvastatin group (HF diet feeding+atorvastatin calcium hydrate administration), and atorvastatin combination group (HF diet feeding+EPA-E administration+atorvastatin calcium hydrate administration) are set. During the feeding, the EPA-E group is administered with 1000 mg/kg of EPA-E; the acetylsalicylic acid group is administered with 10 mg/kg of acetylsalicylic acid; the acetylsalicylic acid combination group is administered with 1000 mg/kg of EPA-E and 10 mg/kg of acetylsalicylic acid; the atorvastatin group is administered with 10 mg/kg of atorvastatin calcium hydrate; and the atorvastatin combination group is administered with 1000 mg/kg of EPA-E and 10 mg/kg of atorvastatin calcium hydrate, after suspending in 5% aqueous solution of gum arabic. The administration is conducted orally once a day. The normal group and the control group are orally administered with 5% aqueous solution of gum arabic once a day. After 4 weeks, blood is collected for determining neutrophil count and conduct biochemical tests of the plasma.

Compared to the normal group, the control group has significantly increased plasma AST and ALT. The neutrophil count, TNFα, IL-6, and high sensitivity CPR as well as ferritin, thioredoxin, and type IV collagen also increase.

Compared to the control group, the EPA-E group and the atorvastatin group exhibit suppressed increase of the plasma AST and ALT, and also, suppressed increase of the neutrophil count, TNFα, IL-6, high sensitivity CPR, ferritin, thioredoxin, and type IV collagen. In contrast, the acetylsalicylic acid group does not exhibit significant improvement over the control group. With regard to the effects of improving the parameters, the acetylsalicylic acid combination group and the atorvastatin combination group exhibit effects which are larger than the sum of the effects realized in each of the EPA-E group, the acetylsalicylic acid group, and the atorvastatin group. Accordingly, the prophylactic/ameliorating or therapeutic agent of the present invention is useful for prevention/amelioration or treatment of the NASH.

### Experimental Example 4

Patients with the definitive diagnosis of NASH are divided into 8 groups (each group consisting of 15 patients). EPA-E group is administered with Epadel S (Registered Trademark) 900 (containing 900 mg of EPA-E) twice a day. Metformin group is administered with Melbin (Registered Trademark) tablet (containing 250 mg of metformin hydrochloride), and metformin combination group is administered with Melbin (Registered Trademark) tablet and Epadel S (Registered Trademark) 900 twice a day. Administration of the Melbin (Registered Trademark) tablet is started from once a day, the dose is adequately adjusted depending on the conditions of the patient, and after 5 weeks from the start of the administration, the dose may be increased to twice a day, and after 9 weeks from the start of the administration, the dose may be increased to 3 times a day. The dose is adequately adjusted depending on the conditions of the patient. Acetylsalicylic acid combination group is administered with Epadel S (Registered Trademark) 900 and Bayaspirin (Registered Trademark) tablet (containing 100 mg of acetylsalicylic acid) twice a day. Administration of the Bayaspirin (Registered Trademark) tablet is started from twice a day at 10 tablet in total, and after 5 weeks from the start of the administration, the dose is reduced to once a day at 1 tablet in total. The dose is adequately adjusted depending on the conditions of the patient. Pravastatin group is administered with Mevalotin (Registered Trademark) tablet 5 (containing 5 mg of pravastatin sodium salt) once a day; pravastatin combination group is administered with Mevalotin (Registered Trademark) tablet 5 once a day and Epadel S (Registered Trademark) 900 twice a day. Candesartan group is administered with Blopress (Registered Trademark) tablet 2 (containing 2 mg of candesartan cilexetil), and candesartan combination group is administered with Blopress (Registered Trademark) tablet 2 and Epadel S (Registered Trademark) 900 twice a day. Administration of the Mevalotin (Registered Trademark) tablet 5 and Blopress (Registered Trademark) tablet 2 is started from 1 tablet once a day, and after 5 weeks from the start of the administration, the dose is increased to twice a day at 2 tablets in total, and after 9 weeks from the start of the administration, the dose is further increased to twice a day at 4 tablets in total. The dose is adequately adjusted depending on the conditions of the patient. Criteria, monitoring, histological tests, statistical analysis of the patients are conducted according to the methods described in Am. J. Gastroenterol. 2001; 96: 2711-2717. Measurement of blood neutrophil count and HOMA-IR, blood biochemical tests such as ALT and AST are occasionally conducted during the administration period of one year, and liver biopsy is conducted after completing the administration for histological evaluation.

In the NASH patients of the EPA-E group, HOMA-IR improved, and blood neutrophil count and AZT, AST, TNFα, IL-6, high sensitivity CPR, ferritin, thioredoxin, fibrosis marker and other blood biochemical parameters decrease compared to the measurements before the treatment, and adiponectin increases. Pathological test image of the liver tissue improves in overall rating of fat accumulation grade, inflammation grade, and fibrosis stage by Brunt method compared to the condition before the administration. In each combination group, index such as blood ALT and AST and pathological test of the liver tissue synergistically improved. In the metformin combination group, TNFα and adiponectin are synergistically improved, and in the acetylsalicylic acid combination group, TNFα, IL-6, high sensitivity CPR, ferritin, and thioredoxin are synergistically improved, and in the candesartan combination group, HOMA-IR and fibrosis marker are synergistically improved.

In the metformin combination group, increase in the dose of Melbin (Registered Trademark) tablet is reduced, and increase in the plasma lactic acid level is suppressed compared to the metformin group. In the acetylsalicylic acid combination group decrease in the dose of Bayaspirin is larger, but decrease in the dose of the Bayaspirin because of the side effect and the like or drug withdrawal is not noted. In the pravastatin combination group, increase in the dose of the Mevalotin (Registered Trademark) tablet 5 is reduced, and increase in the serum CPK level is suppressed compared to the pravastatin group. In the candesartan combination group, increase in the dose of the Blopress (Registered Trademark) tablet 2 is reduced, and transient hypotension is not noted compared to the candesartan group. Accordingly, the prophylactic/ameliorating or therapeutic agent of the present invention is effective for prevention/amelioration or treatment of NASH, and also, for relief for side effects such as lactic acidosis by the biguanide hypoglycemic agent, relief for side effects such as aspirin hypersensitivity or gastrointestinal disorder by the acetylsalicylic acid, relief for the side effects such as rhabdomyolysis by the HMG-CoA reductase inhibitor, or relief for the side effects such as transient hypotension by the ARB.

Combined drugs of ω3PUFAs and biguanide hypoglycemic agent, NSAIDs, HMG-CoA reductase inhibitor, or ARB are prepared by a method commonly used in the art.

### Preparation Example 1

### Soft Capsule Preparation

TABLE 1
Preparation Example 1: Soft capsule preparation

| Component | Composition | |
|---|---|---|
| EPA-E | 300 | mg |
| Metformin | 40 | mg |
| hydrochloride | | |
| Gelatine | 170 | mg |
| D-sorbitol | 25 | mg |
| Conc-glycerin | 25 | mg |

| | |
|---|---|
| Sodium hydroxide | Adequate amount |
| Purified water | Adequate amount |

Of the ingredients of the composition of Table 1, above, the concentrated glycerin, the metformin hydrochloride, and the purified water are mixed, and after stirring the mixture, the sodium hydroxide is added for adjustment of the pH to approximately 7. The gelatin and the D-sorbitol are added to this solution, and the mixture is heated to 60° C. and stirred for dissolution or homogeneous dispersion. This solution or dispersion is defoamed under reduced pressure, and the purified water is added for viscosity adjustment to thereby obtain the solution for the capsule of the soft capsule preparation. This capsule solution for the soft capsule preparation and EPA-E are used to produce a soft capsule preparation containing 300 mg of EPA-E and 40 mg of metformin hydrochloride per capsule.

The procedure as described above is repeated to prepare the soft capsule preparations by replacing the metformin hydrochloride with 8 mg of buformin hydrochloride, 50 mg of acetylsalicylic acid, 5 mg of simvastatin, 5 mg of lovastatin, 3 mg of pravastatin sodium salt, 5 mg of fluvastatin sodium salt, 3 mg of atorvastatin calcium hydrate, 1 mg of rosuvastatin calcium salt, 0.3 mg of pitavastatin calcium salt, 5 mg of valsartan, 4 mg of losartan potassium salt, 6 mg of irbesartan, 50 mg of eprosartan mesylate, 0.5 mg of candesartan cilexetil, 3 mg of telmisartan, or 1 mg of olmesartan medoxomil.

### Preparation Example 2

### Soft Capsule Preparation

TABLE 2
Preparation Example 2: Soft capsule preparation

| Component | | Composition | |
|---|---|---|---|
| A | EPA-E | 300 | mg |
| | Metformin | 80 | mg |
| | hydrochloride | | |
| B | Gelatine | 170 | mg |
| | D-sorbitol | 25 | mg |
| | Conc-glycerin | 25 | mg |

| | |
|---|---|
| Sodium hydroxide | Adequate amount |
| Purified water | Adequate amount |

Water is added to the concentrated glycerin of the composition of Table 2, B, above, and then the gelatin and the D-sorbitol are added to this solution, and the mixture is heated to 60° C. and stirred for dissolution. This solution is defoamed under reduced pressure, and the purified water is added for viscosity adjustment to thereby obtain the solution for the capsule of the soft capsule preparation. According to the composition A, above, the finely pulverized metformin hydrochloride is added to the EPA-E, and the mixture is stirred for homogeneous dispersion to thereby obtain the solution for the content of the soft capsule preparation. This capsule solution for the soft capsule preparation and the soft capsule preparation content solution are used to produce a soft capsule preparation containing 300 mg of EPA-E and 80 mg of metformin hydrochloride per capsule.

The procedure as described above is repeated to prepare the soft capsule preparations by replacing the metformin hydrochloride with 16 mg of buformin hydrochloride, 10 mg of acetylsalicylic acid, 2 mg of simvastatin, 2 mg of lovastatin, 1 mg of pravastatin sodium salt, 2 mg of fluvastatin sodium salt, 1 mg of atorvastatin calcium hydrate, 0.4 mg of rosuvastatin calcium salt, 0.1 mg of pitavastatin calcium salt, 2 mg of valsartan, 1.5 mg of losartan potassium salt, 2.5 mg of irbesartan, 20 mg of eprosartan mesylate, 0.2 mg of candesartan cilexetil, 1 mg of telmisartan, or 2 mg of olmesartan medoxomil. A soft capsule preparation is also produced using 300 mg of K85EE instead of the EPA-E.

### Preparation Example 3

### Liquid Preparation

TABLE 3
Preparation Example 3: Liquid preparation

| Component | | Composition | |
|---|---|---|---|
| A | EPA-E | 1800 | mg |
| | Orange oil | 81 | mg |
| B | Metformin hydrochloride | 500 | mg |
| | Polyoxyethylene (105) | 144 | mg |
| | polyoxypropylene (5) glycol | | |
| | Trehalose | 1350 | mg |
| | Ascorbic stearate | 1.8 | mg |
| | Sodium erythorbate | 117 | mg |
| C | Sodium hydroxide | Adequate amount | |
| | Purified water | Adequate amount | |
| | Total | 9 | g |

Purified water is added to the ingredients of the composition of Table 3, B, above, for dissolution or homogeneous dispersion, and sodium hydroxide is added to adjust the pH to approximately 7. Ingredients of A are added to this solution, and the mixture is stirred at high speed under reduced pressure. 9 g of the resulting emulsion is dispensed in an aluminum laminate film stick package, and after purging the interior of the package with nitrogen, the package is sealed to prepare a liquid preparation containing 1800 mg of EPA-E and 500 mg of metformin hydrochloride per package.

The procedure as described above is repeated to prepare the soft capsules by replacing the metformin hydrochloride with 100 mg of buformin hydrochloride, 600 mg of acetylsalicylic acid, 10 mg of simvastatin, 40 mg of lovastatin, 10 mg of pravastatin sodium salt, 30 mg of fluvastatin sodium salt, 20 mg of atorvastatin calcium hydrate, 5 mg of rosuvastatin calcium salt, 2 mg of pitavastatin calcium salt, 80 mg of valsartan, 50 mg of losartan potassium salt, 100 mg of irbesartan, 800 mg of eprosartan mesylate, 8 mg of candesartan cilexetil, 40 mg of telmisartan, or 20 mg of olmesartan medoxomil. A soft capsule is also produced using 1800 mg of K85EE instead of the EPA-E.

### Preparation Example 4

### Jelly Preparation

TABLE 4
Preparation Example 4: Jelly preparation

| Component | | Composition | |
|---|---|---|---|
| A | EPA-E | 1800 | mg |
| | Orange oil | 81 | mg |
| B | Metformin hydrochloride | 250 | mg |
| | Polyoxyethylene (105) | 144 | mg |
| | polyoxypropylene (5) glycol | | |
| | Trehalose | 1350 | mg |
| | Ascorbic stearate | 1.8 | mg |
| | Sodium erythorbate | 117 | mg |
| | Pullulan | 270 | mg |
| C | Carrageenan | 37.8 | gm |
| | Carob bean gum | 22.5 | mg |
| | Conc-glycerin | 675 | mg |
| D | Sodium hydroxide | Adequate amount | |
| | Purified water | Adequate amount | |
| | Total | 9 | g |

The purified water is added to the ingredients of the composition of Table 4, B, above, for dissolution or homogeneous dispersion, and the sodium hydroxide is added to adjust the pH to approximately 7. Ingredients of the composition A are added to this solution, and the mixture is stirred at high speed under reduced pressure to form an emulsion. The resulting emulsion is heated to 85° C., and to this emulsion is added a dispersion of the ingredients of the composition C which has been stirred for homogeneous dispersion. The mixture is homogeneously mixed, and 9 g of the resulting preparation is dispensed in an aluminum laminate film stick package, and after purging the interior of the package with nitrogen, the package is sealed and cooled for solidification to thereby prepare a jelly preparation containing 1800 mg of EPA-E and 250 mg of metformin hydrochloride per package.

The procedure as described above is repeated to prepare the jelly preparation by replacing the metformin hydrochloride with 50 mg of buformin hydrochloride, 300 mg of acetylsalicylic acid, 20 mg of simvastatin, 40 mg of lovastatin, 20 mg of pravastatin sodium salt, 30 mg of fluvastatin sodium salt, 20 mg of atorvastatin calcium hydrate, 5 mg of rosuvastatin calcium salt, 2 mg of pitavastatin calcium salt, 40 mg of valsartan, 25 mg of losartan potassium salt, 50 mg of irbesartan, 400 mg of eprosartan mesylate, 4 mg of candesartan cilexetil, 20 mg of telmisartan, or 10 mg of olmesartan medoxomil.

### Preparation Example 5

### Composite Formulation Package

Seamless soft capsules having a gelatin film with the diameter of about 4 mm and containing 20 mg of EPA-E are prepared. 45 capsules of the EPA-E-containing seamless soft capsule, and 1 tablet of Melbin (Registered Trademark) tablet (containing 250 mg of metformin hydrochloride per 280 mg tablet), 3 tablets of Bayaspirin (Registered Trademark) tablet (containing 100 mg of acetylsalicylic acid in 137 mg tablet), 1 tablet of Lochol (Registered Trademark) 10 mg tablet (containing 10 mg of fluvastatin sodium salt in 66 mg tablet), or 1 tablet of Micardis (Registered Trademark) tablet 20 (containing 20 mg of telmisartan in a tablet of about 85 mg) are placed in an aluminum laminate film stick package, and the interior is purged with nitrogen. The package is sealed to prepare a composite formulation package containing 900 mg of EPA-E, and 250 mg of metformin hydrochloride, 100 mg of acetylsalicylic acid, 10 mg of fluvastatin sodium salt, or 20 mg of telmisartan per package. The procedure as described above is repeated by replacing the EPA-E with a seamless capsule containing 20 mg of K85EE to prepare a composite formulation package.

90 capsules of the EPA-E-containing seamless soft capsule and 1 tablet of Glucophage XR (Registered Trademark) 500 mg tablet, 1 tablet of Measurin tablet (containing 650 mg of acetylsalicylic acid per tablet), 1 tablet of Crestar (Registered Trademark) 10 mg tablet (containing 10 mg of rosuvastatin calcium salt in 150 mg tablet), or 1 tablet of Nu-Lotan (Registered Trademark) tablet 50 (containing 50 mg of losartan potassium salt per tablet) 1 tablet are placed in an aluminum laminate film stick package, and the interior is purged with nitrogen. The package is sealed to prepare a composite formulation package containing 1800 mg of EPA-E, and 500 mg of metformin hydrochloride, 650 mg of acetylsalicylic acid, 10 mg of rosuvastatin calcium salt, or 50 mg of losartan potassium salt.

### INDUSTRIAL APPLICABILITY

The prophylactic/ameliorating or therapeutic agent for NASH of the present invention containing a combination of at least one first ingredient selected from the group consisting of ω3 PUFAs and pharmaceutically acceptable salts and esters thereof and at least one second ingredient selected from the group consisting of biguanide hypoglycemic agents, NSAIDs, HMG-CoA reductase inhibitors and ARBs as the active ingredients is expected to exhibit synergetic prophylactic/ameliorative or therapeutic effects for the NASH compared to the single use of the corresponding agents.

For example, when the second ingredient is a biguanide, the prophylactic/ameliorating or therapeutic agent of the present invention is expected to specifically exhibit synergistic prophylactic/ameliorative or therapeutic effects in NASH patients who have developed insulin resistance, NASH patients with reduced adiponectin, or NASH patients with increased TNFα including the patients suffering from diabetes or metabolic syndrome. The drug is also expected to bring patients not simultaneously suffering from the hyperlipemia a marked amelioration in their pathological conditions by the combined use with the small amount of the biguanide hypoglycemic agent.

When the second ingredient is an HMG-CoA reductase inhibitor, the prophylactic/ameliorating or therapeutic agent of the present invention is expected to specifically exhibit synergistic prophylactic/ameliorative or therapeutic effects in NASH patients who have developed insulin resistance, NASH patients with increased TNFα, IL, or high sensitivity CRP, or NASH patients with increased fibrosis marker (such as type IV collagen, hyaluronic acid, or TIMP-1) or blood oxidative stress marker (such as ferritin and thioredoxin) including the patients suffering from hyperlipidemia or metabolic syndrome. The drug is also expected to bring patients not simultaneously suffering from the hyperlipidemia a marked amelioration in their pathological conditions by the combined use with the small amount of the HMG-CoA reductase inhibitor.

When the second ingredient is an ARB, the prophylactic/ameliorating or therapeutic agent of the present invention is expected to specifically exhibit synergistic prophylactic/ameliorative or therapeutic effects in NASH patients who have developed insulin resistance with increased fibrosis marker (such as type IV collagen, hyaluronic acid, or TIMP-1) or blood oxidative stress marker (such as ferritin and thioredoxin) including the patients suffering from hypertension or metabolic syndrome. The drug is also expected to bring patients not simultaneously suffering from the hypertension a marked amelioration in their pathological conditions by the combined use with the small amount of the ARB.

The present invention enables administration at a reduced dose of the active ingredients, and in particular, administration at a reduced dose of the second ingredient when used alone, and this results in relieving the side effects. Decrease in the dose also enables treatment of the patients who could not receive the treatment or the patients who had to stop the treatment because of the side effects.

For example, the treatment can be continued in patients and more specifically, in patients who have the risk of suffering from side effects by the biguanide hypoglycemic agent such as lactic acidosis or delayed hypoglycemia; patient who had to stop the acetylsalicylic acid administration due to the side effects such as aspirin hypersensitivity and gastrointestinal disorder induced by acetylsalicylic acid; patients suffering from angina pectoris, myocardial infarction, or ischemic or cerebrovascular disease, and patients who have experienced coronary artery bypass grafting or percutaneous transluminal coronary angioplasty who are in need of continued administration of the acetylsalicylic acid; patients who have the risk of rhabdomyolysis which is a serious side effect of HMG-CoA reductase inhibitor; and patients who could not receive the ARB or patients who had to stop the treatment because of the side effects, such as patients who have the risk of suffering from side effects by the ARB such as renal dysfunction, hepatic dysfunction, or shock symptoms by transient hypotension.

Furthermore, the present invention is capable of further improving the prophylactic/ameliorative or therapeutic effects by reducing the burden of the patients by providing the agent in the form of a composite formulation or a kit and thereby improving the drug compliance.

1. A prophylactic/ameliorating or therapeutic agent for non-alcoholic steatohepatitis, in which at least one first ingredient selected from the group consisting of an ω3 polyunsaturated fatty acid and pharmaceutically acceptable salts and esters thereof, and at least one second ingredient selected from the group consisting of
(a) a biguanide hypoglycemic agent,
(b) a nonsteroidal anti-inflammatory drug,
(c) a 3-hydroxy-3-methyl glutaryl coenzyme A reductase inhibitor, and
(d) an angiotensin II receptor blocker are applied in combination as active ingredients.

2. A prophylactic/ameliorating or therapeutic agent according to claim 1 wherein the first active ingredient is at least one compound selected from the group consisting of icosapentaenoic acid, docosahexaenoic acid, α-linolenic acid, and pharmaceutically acceptable salts and esters thereof.

3. A prophylactic/ameliorating or therapeutic agent according to claim 1 or 2 wherein the agent is a composite formulation of the first active ingredient and the second active ingredient.

4. A prophylactic/ameliorating or therapeutic agent according to any one of claims 1 to 3 wherein the second active ingredient is a biguanide hypoglycemic agent (a).

5. A prophylactic/ameliorating or therapeutic agent according to any one of claims 1 to 4 wherein the biguanide hypoglycemic agent (a) is at least one compound selected from the group consisting of metformin, buformin, phenformin, and pharmaceutically acceptable salts thereof.

6. A prophylactic/ameliorating or therapeutic agent according to any one of claims 1 to 3 wherein the second active ingredient (b) is a nonsteroidal anti-inflammatory drug.

7. A prophylactic/ameliorating or therapeutic agent according to claim 6 wherein the nonsteroidal anti-inflammatory drug (b) is at least one member selected from the group consisting of acetylsalicylic acid and pharmaceutically acceptable salts and esters thereof.

8. A prophylactic/ameliorating or therapeutic agent according to any one of claims 1 to 3 wherein the second active ingredient is a 3-hydroxy-3-methyl glutaryl coenzyme A reductase inhibitor (c).

9. A prophylactic/ameliorating or therapeutic agent according to claim 8 wherein the 3-hydroxy-3-methyl glutaryl coenzyme A reductase inhibitor is at least one compound selected from the group consisting of lovastatin, pravastatin, simvastatin, fluvastatin, atorvastatin, rosuvastatin, pitavastatin, cerivastatin, and pharmaceutically acceptable salts thereof.

10. A prophylactic/ameliorating or therapeutic agent according to any one of claims 1 to 3 wherein the second active ingredient an angiotensin II receptor blocker (d).

11. A prophylactic/ameliorating or therapeutic agent according to claim 10 wherein the angiotensin IT receptor blocker is at least one compound selected from the group consisting of losartan, valsartan, irbesartan, eprosartan, candesartan, telmisartan, olmesartan, and pharmaceutically acceptable salts thereof.
